(19) United States
(12) Patent Application Publication
(10) Pub. No.: US 2008/0000645 A1
(43) Pub. Date: Jan. 3, 2008

(54) Radial Expansion System

(75) Inventors: David Paul Brisco, Duncan, OK (US); Brock Wayne Watson, Carrollton, TX (US); Mark Shuster, Rozensoomlaan (NL); Malcolm Gray, Houston, TX (US); Grigoriy Grinberg, Sylvania, OH (US); Scott Costa, Katy, TX (US); Russell Wasson, Bourbon, MO (US)

Correspondence Address: HAYNES AND BOONE, LLP, 901 MAIN STREET, SUITE 3100, DALLAS, TX, 75202-3789, US

(73) Assignee: ENVENTURE GLOBAL TECHNOLOGY, LLC, Houston, TX (US)

(21) Appl. No.: 11/573,309

(22) PCT Filed: Aug. 11, 2005
(86) PCT No.: PCT/US05/28642

§ 371 Date: Feb. 6, 2007

Related U.S. Application Data
(60) Provisional application No. 60/600,679, filed on Aug. 11, 2004.

(51) Int. Cl. E21B 19/16; G01B 7/16; G01N 33/00

(52) U.S. Cl. 166/380; 436/145; 73/760

(57) ABSTRACT

A method of radially expanding and plastically deforming tubular members (10, 100, 200) is provided that includes selecting a tubular member, determining an anisotropy value and a strain hardening value for the selected tubular member (10, 100, 200), multiplying the anisotropy value times the strain hardening value to generate an expandability value for the selected tubular member (10, 100, 200); and if the expandability value is greater than 0.12, then radially expanding and plastically deforming the selected tubular member (10, 100, 200).

### CROSS REFERENCE TO RELATED APPLICATIONS

This application claims the benefit of the filing date of U.S. provisional patent application Ser. No. 60/600,679, attorney docket number 25791.194, filed on Aug. 11, 2004, the disclosure which is incorporated herein by reference.

This application is a continuation-in-part of one or more of the following: (1) PCT application US02/04353, filed on Feb. 14, 2002, attorney docket no. 25791.50.02, which claims priority from U.S. provisional patent application Ser. No. 60/270,007, attorney docket no. 25791.50, filed on Feb. 20, 2001; (2) PCT application US 03/00609, filed on Jan. 9, 2003, attorney docket no. 25791.71.02, which claims priority from U.S. provisional patent application Ser. No. 60/357,372, attorney docket no. 25791.71, filed on Feb. 15, 2002; and (3) U.S. provisional patent application Ser. No. 60/585,370, attorney docket number 25791.299, filed on Jul. 2, 2004, the disclosures of which are incorporated herein by reference.

This application is related to the following co-pending applications: (1) U.S. Pat. No. 6,497,289, which was filed as U.S. patent application Ser. No. 09/454,139, attorney docket no. 25791.03.02, filed on Dec. 3, 1999, which claims priority from provisional application 60/111,293, filed on Dec. 7, 1998, (2) U.S. patent application Ser. No. 09/510,913, attorney docket no. 25791.7.02, filed on Feb. 23, 2000, which claims priority from provisional application 60/121,702, filed on Feb. 25, 1999, (3) U.S. patent application Ser. No. 09/502,350, attorney docket no. 25791.8.02, filed on Feb. 10, 2000, which claims priority from provisional application 60/119,611, filed on Feb. 11, 1999, (4) U.S. Pat. No. 6,328,113, which was filed as U.S. patent application Ser. No. 09/440,338, attorney docket number 25791.9.02, filed on Nov. 15, 1999, which claims priority from provisional application 60/108,558, filed on Nov. 16, 1998, (5) U.S. patent application Ser. No. 10/169,434, attorney docket no. 25791.10.04, filed on Jul. 1, 2002, which claims priority from provisional application 60/183,546, filed on Feb. 18, 2000, (6) U.S. patent application Ser. No. 09/523,468, attorney docket no. 25791.11.02, filed on Mar. 10, 2000, which claims priority from provisional application 60/124,042, filed on Mar. 11, 1999, (7) U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (8) U.S. Pat. No. 6,575,240, which was filed as patent application Ser. No. 09/511,941, attorney docket no. 25791.16.02, filed on Feb. 24, 2000, which claims priority from provisional application 60/121,907, filed on Feb. 26, 1999, (9) U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (10) U.S. patent application Ser. No. 09/981,916, attorney docket no. 25791.18, filed on Oct. 18, 2001 as a continuation-in-part application of U.S. Pat. No. 6,328,113, which was filed as U.S. patent application Ser. No. 09/440,338, attorney docket number 25791.9.02, filed on Nov. 15, 1999, which claims priority from provisional application 60/108,558, filed on Nov. 16, 1998, (11) U.S. Pat. No. 6,604,763, which was filed as application Ser. No. 09/559,122, attorney docket no. 25791.23.02, filed on Apr. 26, 2000, which claims priority from provisional application 60/131,106, filed on Apr. 26, 1999, (12) U.S. patent application Ser. No. 10/030,593, attorney docket no. 25791.25.08, filed on Jan. 8, 2002, which claims priority from provisional application 60/146,203, filed on Jul. 29, 1999, (13) U.S. provisional patent application Ser. No. 60/143,039, attorney docket no. 25791.26, filed on Jul. 9, 1999, (14) U.S. patent application Ser. No. 10/111,982, attorney docket no. 25791.27.08, filed on Apr. 30, 2002, which claims priority from provisional patent application Ser. No. 60/162,671, attorney docket no. 25791.27, filed on Nov. 1, 1999, (15) U.S. provisional patent application Ser. No. 60/154,047, attorney docket no. 25791.29, filed on Sep. 16, 1999, (16) U.S. provisional patent application Ser. No. 60/438,828, attorney docket no. 25791.31, filed on Jan. 9, 2003, (17) U.S. Pat. No. 6,564,875, which was filed as application Ser. No. 09/679,907, attorney docket no. 25791.34.02, on Oct. 5, 2000, which claims priority from provisional patent application Ser. No. 60/159,082, attorney docket no. 25791.34, filed on Oct. 12, 1999, (18) U.S. patent application Ser. No. 10/089,419, filed on Mar. 27, 2002, attorney docket no. 25791.36.03, which claims priority from provisional patent application Ser. No. 60/159,039, attorney docket no. 25791.36, filed on Oct. 12, 1999, (19) U.S. patent application Ser. No. 09/679,906, filed on Oct. 5, 2000, attorney docket no. 25791.37.02, which claims priority from provisional patent application Ser. No. 60/159,033, attorney docket no. 25791.37, filed on Oct. 12, 1999, (20) U.S. patent application Ser. No. 10/303,992, filed on Nov. 22, 2002, attorney docket no. 25791.38.07, which claims priority from provisional patent application Ser. No. 60/212,359, attorney docket no. 25791.38, filed on Jun. 19, 2000, (21) U.S. provisional patent application Ser. No. 60/165,228, attorney docket no. 25791.39, filed on Nov. 12, 1999, (22) U.S. provisional patent application Ser. No. 60/455,051, attorney docket no. 25791.40, filed on Mar. 14, 2003, (23) PCT application US02/2477, filed on Jun. 26, 2002, attorney docket no. 25791.44.02, which claims priority from U.S. provisional patent application Ser. No. 60/303,711, attorney docket no. 25791.44, filed on Jul. 6, 2001, (24) U.S. patent application Ser. No. 10/311,412, filed on Dec. 12, 2002, attorney docket no. 25791.45.07, which claims priority from provisional patent application Ser. No. 60/221,443, attorney docket no 25791.45, filed on Jul. 28, 2000, (25) U.S. patent application Ser. No. 10/, filed on Dec. 18, 2002, attorney docket no. 25791.46.07, which claims priority from provisional patent application Ser. No. 60/221,645, attorney docket no. 25791.46, filed on Jul. 28, 2000, (26) U.S. patent application Ser. No. 10/322,947, filed on Jan. 22, 2003, attorney docket no. 25791.47.03, which claims priority from provisional patent application Ser. No. 60/233,638, attorney docket no. 25791.47, filed on Oct. 18, 2000, (27) U.S. patent application Ser. No. 10/406,648, filed on Mar. 31, 2003, attorney docket no. 25791.48.06, which claims priority from provisional patent application Ser. No. 60/237,334, attorney docket no. 25791.48, filed on Oct. 2, 2000, (28) PCT application US02/04353, filed on Feb. 14, 2002, attorney docket no. 25791.50.02, which claims priority from U.S. provisional patent application Ser. No. 60/270,007, attorney docket no. 25791.50, filed on Feb. 20, 2001, (29) U.S. patent application Ser. No. 10/465,835, filed on Jun. 13, 2003, attorney docket no. 25791.51.06, which claims priority from provisional patent application Ser. No. 60/262,434, attorney docket no. 25791.51, filed on Jan. 17, 2001, (30) U.S. patent application Ser. No. 10/465,831, filed on Jun. 13, 2003, attorney docket no. 25791.52.06, which claims priority from U.S. provisional patent application Ser. No. 60/259,486, attorney docket no. 25791.52, filed on Jan. 3, 2001, (31) U.S. provisional patent application Ser. No. 60/452,303, filed on Mar. 5, 2003, attorney docket no. 25791.53, (32) U.S. Pat. No. 6,470,966, which was filed as patent application Ser. No. 09/850,093, filed on May 7, 2001, attorney docket no. 25791.55, as a divisional application of U.S. Pat. No. 6,497,289, which was filed as U.S. patent application Ser. No. 09/454,139, attorney docket no. 25791.03.02, filed on Dec. 3, 1999, which claims priority from provisional application 60/111,293, filed on Dec. 7, 1998, (33) U.S. Pat. No. 6,561,227, which was filed as patent application Ser. No. 09/852,026, filed on May 9, 2001, attorney docket no. 25791.56, as a divisional application of U.S. Pat. No. 6,497,289, which was filed as U.S. patent application Ser. No. 09/454,139, attorney docket no. 25791.03.02, filed on Dec. 3, 1999, which claims priority from provisional application 60/111,293, filed on Dec. 7, 1998, (34) U.S. patent application Ser. No. 09/852,027, filed on May 9, 2001, attorney docket no. 25791.57, as a divisional application of U.S. Pat. No. 6,497,289, which was filed as U.S. patent application Ser. No. 09/454,139, attorney docket no. 25791.03.02, filed on Dec. 3, 1999, which claims priority from provisional application 60/111,293, filed on Dec. 7, 1998, (35) PCT Application US02/25608, attorney docket no. 25791.58.02, filed on Aug. 13, 2002, which claims priority from provisional application 60/318,021, filed on Sep. 7, 2001, attorney docket no. 25791.58, (36) PCT Application US02/24399, attorney docket no. 25791.59.02, filed on Aug. 1, 2002, which claims priority from U.S. provisional patent application Ser. No. 60/313,453, attorney docket no. 25791.59, filed on Aug. 20, 2001, (37) PCT Application US02/29856, attorney docket no. 25791.60.02, filed on Sep. 19, 2002, which claims priority from U.S. provisional patent application Ser. No. 60/326,886, attorney docket no. 25791.60, filed on Oct. 3, 2001, (38) PCT Application US02/20256, attorney docket no. 25791.61.02, filed on Jun. 26, 2002, which claims priority from U.S. provisional patent application Ser. No. 60/303,740, attorney docket no. 25791.61, filed on Jul. 6, 2001, (39) U.S. patent application Ser. No. 09/962,469, filed on Sep. 25, 2001, attorney docket no. 25791.62, which is a divisional of U.S. patent application Ser. No. 09/523,468, attorney docket no. 25791.11.02, filed on Mar. 10, 2000, which claims priority from provisional application 60/124,042, filed on Mar. 11, 1999, (40) U.S. patent application Ser. No. 09/962,470, filed on Sep. 25, 2001, attorney docket no. 25791.63, which is a divisional of U.S. patent application Ser. No. 09/523,468, attorney docket no. 25791.11.02, filed on Mar. 10, 2000, which claims priority from provisional application 60/124,042, filed on Mar. 11, 1999, (41) U.S. patent application Ser. No. 09/962,471, filed on Sep. 25, 2001, attorney docket no. 25791.64, which is a divisional of U.S. patent application Ser. No. 09/523,468, attorney docket no. 25791.11.02, filed on Mar. 10, 2000, which claims priority from provisional application 60/124,042, filed on Mar. 11, 1999, (42) U.S. patent application Ser. No. 09/962,467, filed on Sep. 25, 2001, attorney docket no. 25791.65, which is a divisional of U.S. patent application Ser. No. 09/523,468, attorney docket no. 25791.11.02, filed on Mar. 10, 2000, which claims priority from provisional application 60/124,042, filed on Mar. 11, 1999, (43) U.S. patent application Ser. No. 09/962,468, filed on Sep. 25, 2001, attorney docket no. 25791.66, which is a divisional of U.S. patent application Ser. No. 09/523,468, attorney docket no. 25791.11.02, filed on Mar. 10, 2000, which claims priority from provisional application 60/124,042, filed on Mar. 11, 1999, (44) PCT application US 02/25727, filed on Aug. 14, 2002, attorney docket no. 25791.67.03, which claims priority from U.S. provisional patent application Ser. No. 60/317,985, attorney docket no. 25791.67, filed on Sep. 6, 2001, and U.S. provisional patent application Ser. No. 60/318,386, attorney docket no. 25791.67.02, filed on Sep. 10, 2001, (45) PCT application US 02/39425, filed on Dec. 10, 2002, attorney docket no. 25791.68.02, which claims priority from U.S. provisional patent application Ser. No. 60/343,674, attorney docket no. 25791.68, filed on Dec. 27, 2001, (46) U.S. utility patent application Ser. No. 09/969,922, attorney docket no. 25791.69, filed on Oct. 3, 2001, which is a continuation-in-part application of U.S. Pat. No. 6,328,113, which was filed as U.S. patent application Ser. No. 09/440,338, attorney docket number 25791.9.02, filed on Nov. 15, 1999, which claims priority from provisional application 60/108,558, filed on Nov. 16, 1998, (47) U.S. utility patent application Ser. No. 10/516,467, attorney docket no. 25791.70, filed on Dec. 10, 2001, which is a continuation application of U.S. utility patent application Ser. No. 09/969,922, attorney docket no. 25791.69, filed on Oct. 3, 2001, which is a continuation-in-part application of U.S. Pat. No. 6,328,113, which was filed as U.S. patent application Ser. No. 09/440,338, attorney docket number 25791.9.02, filed on Nov. 15, 1999, which claims priority from provisional application 60/108,558, filed on Nov. 16, 1998, (48) PCT application US 03/00609, filed on Jan. 9, 2003, attorney docket no. 25791.71.02, which claims priority from U.S. provisional patent application Ser. No. 60/357,372, attorney docket no. 25791.71, filed on Feb. 15, 2002, (49) U.S. patent application Ser. No. 10/074,703, attorney docket no. 25791.74, filed on Feb. 12, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (50) U.S. patent application Ser. No. 10/074,244, attorney docket no. 25791.75, filed on Feb. 12, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (51) U.S. patent application Ser. No. 10/076,660, attorney docket no. 25791.76, filed on Feb. 15, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (52) U.S. patent application Ser. No. 10/076,661, attorney docket no. 25791.77, filed on Feb. 15, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (53) U.S. patent application Ser. No. 10/076,659, attorney docket no. 25791.78, filed on Feb. 15, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (54) U.S. patent application Ser. No. 10/078,928, attorney docket no. 25791.79, filed on Feb. 20, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (55) U.S. patent application Ser. No. 10/078,922, attorney docket no. 25791.80, filed on Feb. 20, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (56) U.S. patent application Ser. No. 10/078,921, attorney docket no. 25791.81, filed on Feb. 20, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (57) U.S. patent application Ser. No. 10/261,928, attorney docket no. 25791.82, filed on Oct. 1, 2002, which is a divisional of U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (58) U.S. patent application Ser. No. 10/079,276, attorney docket no. 25791.83, filed on Feb. 20, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (59) U.S. patent application Ser. No. 10/262,009, attorney docket no. 25791.84, filed on Oct. 1, 2002, which is a divisional of U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (60) U.S. patent application Ser. No. 10/092,481, attorney docket no. 25791.85, filed on Mar. 7, 2002, which is a divisional of U.S. Pat. No. 6,568,471, which was filed as patent application Ser. No. 09/512,895, attorney docket no. 25791.12.02, filed on Feb. 24, 2000, which claims priority from provisional application 60/121,841, filed on Feb. 26, 1999, (61) U.S. patent application Ser. No. 10/261,926, attorney docket no. 25791.86, filed on Oct. 1, 2002, which is a divisional of U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (62) PCT application US 02/36157, filed on Nov. 12, 2002, attorney docket no. 25791.87.02, which claims priority from U.S. provisional patent application Ser. No. 60/338,996, attorney docket no. 25791.87, filed on Nov. 12, 2001, (63) PCT application US 02/36267, filed on Nov. 12, 2002, attorney docket no. 25791.88.02, which claims priority from U.S. provisional patent application Ser. No. 60/339,013, attorney docket no. 25791.88, filed on Nov. 12, 2001, (64) PCT application US 03/11765, filed on Apr. 16, 2003, attorney docket no. 25791.89.02, which claims priority from U.S. provisional patent application Ser. No. 60/383,917, attorney docket no. 25791.89, filed on May 29, 2002, (65) PCT application US 03/15020, filed on May 12, 2003, attorney docket no. 25791.90.02, which claims priority from U.S. provisional patent application Ser. No. 60/391,703, attorney docket no. 25791.90, filed on Jun. 26, 2002, (66) PCT application US 02/39418, filed on Dec. 10, 2002, attorney docket no. 25791.92.02, which claims priority from U.S. provisional patent application Ser. No. 60/346,309, attorney docket no. 25791.92, filed on Jan. 7, 2002, (67) PCT application US 03/06544, filed on Mar. 4, 2003, attorney docket no. 25791.93.02, which claims priority from U.S. provisional patent application Ser. No. 60/372,048, attorney docket no. 25791.93, filed on Apr. 12, 2002, (68) U.S. patent application Ser. No. 10/331,718, attorney docket no. 25791.94, filed on Dec. 30, 2002, which is a divisional U.S. patent application Ser. No. 09/679,906, filed on Oct. 5, 2000, attorney docket no. 25791.37.02, which claims priority from provisional patent application Ser. No. 60/159,033, attorney docket no. 25791.37, filed on Oct. 12, 1999, (69) PCT application US 03/04837, filed on Feb. 29, 2003, attorney docket no. 25791.95.02, which claims priority from U.S. provisional patent application Ser. No. 60/363,829, attorney docket no. 25791.95, filed on Mar. 13, 2002, (70) U.S. patent application Ser. No. 10/261,927, attorney docket no. 25791.97, filed on Oct. 1, 2002, which is a divisional of U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (71) U.S. patent application Ser. No. 10/262,008, attorney docket no. 25791.98, filed on Oct. 1, 2002, which is a divisional of U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (72) U.S. patent application Ser. No. 10/261,925, attorney docket no. 25791.99, filed on Oct. 1, 2002, which is a divisional of U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (73) U.S. patent application Ser. No. 10/199,524, attorney docket no. 25791.100, filed on Jul. 19, 2002, which is a continuation of U.S. Pat. No. 6,497,289, which was filed as U.S. patent application Ser. No. 09/454,139, attorney docket no. 25791.03.02, filed on Dec. 3, 1999, which claims priority from provisional application 60/111,293, filed on Dec. 7, 1998, (74) PCT application US 03/10144, filed on Mar. 28, 2003, attorney docket no. 25791.101.02, which claims priority from U.S. provisional patent application Ser. No. 60/372,632, attorney docket no. 25791.101, filed on Apr. 15, 2002, (75) U.S. provisional patent application Ser. No. 60/412,542, attorney docket no. 25791.102, filed on Sep. 20, 2002, (76) PCT application US 03/14153, filed on May 6, 2003, attorney docket no. 25791.104.02, which claims priority from U.S. provisional patent application Ser. No. 60/380,147, attorney docket no. 25791.104, filed on May 6, 2002, (77) PCT application US 03/19993, filed on Jun. 24, 2003, attorney docket no. 25791.106.02, which claims priority from U.S. provisional patent application Ser. No. 60/397,284, attorney docket no. 25791.106, filed on Jul. 19, 2002, (78) PCT application US 03/13787, filed on May 5, 2003, attorney docket no. 25791.107.02, which claims priority from U.S. provisional patent application Ser. No. 60/387,486, attorney docket no. 25791.107, filed on Jun. 10, 2002, (79) PCT application US 03/18530, filed on Jun. 11, 2003, attorney docket no. 25791.108.02, which claims priority from U.S. provisional patent application Ser. No. 60/387,961, attorney docket no. 2579.1.108, filed on Jun. 12, 2002, (80) PCT application US 03/20694, filed on Jul. 1, 2003, attorney docket no. 25791.110.02, which claims priority from U.S. provisional patent application Ser. No. 60/398,061, attorney docket no. 25791.110, filed on Jul. 24, 2002, (81) PCT application US 03/20870, filed on Jul. 2, 2003, attorney docket no. 25791.111.02, which claims priority from U.S. provisional patent application Ser. No. 60/399,240, attorney docket no. 25791.111, filed on Jul. 29, 2002, (82) U.S. provisional patent application Ser. No. 60/412,487, attorney docket no. 25791.112, filed on Sep. 20, 2002, (83) U.S. provisional patent application Ser. No. 60/412,488, attorney docket no. 25791.114, filed on Sep. 20, 2002, (84) U.S. patent application Ser. No. 10/280,356, attorney docket no. 25791.115, filed on Oct. 25, 2002, which is a continuation of U.S. Pat. No. 6,470,966, which was filed as patent application Ser. No. 09/850,093, filed on May 7, 2001, attorney docket no. 25791.55, as a divisional application of U.S. Pat. No. 6,497,289, which was filed as U.S. patent application Ser. No. 09/454,139, attorney docket no. 25791.03.02, filed on Dec. 3, 1999, which claims priority from provisional application 60/111,293, filed on Dec. 7, 1998, (85) U.S. provisional patent application Ser. No. 60/412,177, attorney docket no. 25791.117, filed on Sep. 20, 2002, (86) U.S. provisional patent application Ser. No. 60/412,653, attorney docket no. 25791.118, filed on Sep. 20, 2002, (87) U.S. provisional patent application Ser. No. 60/405,610, attorney docket no. 25791.119, filed on Aug. 23, 2002, (88) U.S. provisional patent application Ser. No. 60/405,394, attorney docket no. 25791.120, filed on Aug. 23, 2002, (89) U.S. provisional patent application Ser. No. 60/412,544, attorney docket no. 25791.121, filed on Sep. 20, 2002, (90) PCT application US 03/24779, filed on Aug. 8, 2003, attorney docket no. 25791.125.02, which claims priority from U.S. provisional patent application Ser. No. 60/407,442, attorney docket no. 25791.125, filed on Aug. 30, 2002, (91) U.S. provisional patent application Ser. No. 60/423,363, attorney docket no. 25791.126, filed on Dec. 10, 2002, (92) U.S. provisional patent application Ser. No. 60/412,196, attorney docket no. 25791.127, filed on Sep. 20, 2002, (93) U.S. provisional patent application Ser. No. 60/412,187, attorney docket no. 25791.128, filed on Sep. 20, 2002, (94) U.S. provisional patent application Ser. No. 60/412,371, attorney docket no. 25791.129, filed on Sep. 20, 2002, (95) U.S. patent application Ser. No. 10/382,325, attorney docket no. 25791.145, filed on Mar. 5, 2003, which is a continuation of U.S. Pat. No. 6,557,640, which was filed as patent application Ser. No. 09/588,946, attorney docket no. 25791.17.02, filed on Jun. 7, 2000, which claims priority from provisional application 60/137,998, filed on Jun. 7, 1999, (96) U.S. patent application Ser. No. 10/624,842, attorney docket no. 25791.151, filed on Jul. 22, 2003, which is a divisional of U.S. patent application Ser. No. 09/502,350, attorney docket no. 25791.8.02, filed on Feb. 10, 2000, which claims priority from provisional application 60/119,611, filed on Feb. 11, 1999, (97) U.S. provisional patent application Ser. No. 60/431,184, attorney docket no. 25791.157, filed on Dec. 5, 2002, (98) U.S. provisional patent application Ser. No. 60/448,526, attorney docket no. 25791.185, filed on Feb. 18, 2003, (99) U.S. provisional patent application Ser. No. 60/461,539, attorney docket no. 25791.186, filed on Apr. 9, 2003, (100) U.S. provisional patent application Ser. No. 60/462,750, attorney docket no. 25791.193, filed on Apr. 14, 2003, (101) U.S. provisional patent application Ser. No. 60/436,106, attorney docket no. 25791.200, filed on Dec. 23, 2002, (102) U.S. provisional patent application Ser. No. 60/442,942, attorney docket no. 25791.213, filed on Jan. 27, 2003, (103) U.S. provisional patent application Ser. No. 60/442,938, attorney docket no. 25791.225, filed on Jan. 27, 2003, (104) U.S. provisional patent application Ser. No. 60/418,687, attorney docket no. 25791.228, filed on Apr. 18, 2003, (105) U.S. provisional patent application Ser. No. 60/454,896, attorney docket no. 25791.236, filed on Mar. 14, 2003, (106) U.S. provisional patent application Ser. No. 60/450,504, attorney docket no. 25791.238, filed on Feb. 26, 2003, (107) U.S. provisional patent application Ser. No. 60/451,152, attorney docket no. 25791.239, filed on Mar. 9, 2003, (108) U.S. provisional patent application Ser. No. 60/455,124, attorney docket no. 25791.241, filed on Mar. 17, 2003, (109) U.S. provisional patent application Ser. No. 60/453,678, attorney docket no. 25791.253, filed on Mar. 11, 2003, (110) U.S. patent application Ser. No. 10/421,682, attorney docket no. 25791.256, filed on Apr. 23, 2003, which is a continuation of U.S. patent application Ser. No. 09/523,468, attorney docket no. 25791.11.02, filed on Mar. 10, 2000, which claims priority from provisional application 60/124,042, filed on Mar. 11, 1999, (111) U.S. provisional patent application Ser. No. 60/457,965, attorney docket no. 25791.260, filed on Mar. 27, 2003, (112) U.S. provisional patent application Ser. No. 60/455,718, attorney docket no. 25791.262, filed on Mar. 18, 2003, (113) U.S. Pat. No. 6,550,821, which was filed as patent application Ser. No. 09/811,734, filed on Mar. 19, 2001, (114) U.S. patent application Ser. No. 10/436,467, attorney docket no. 25791.268, filed on May 12, 2003, which is a continuation of U.S. Pat. No. 6,604,763, which was filed as application Ser. No. 09/559,122, attorney docket no. 25791.23.02, filed on Apr. 26, 2000, which claims priority from provisional application 60/131,106, filed on Apr. 26, 1999, (115) U.S. provisional patent application Ser. No. 60/459,776, attorney docket no. 25791.270, filed on Apr. 2, 2003, (116) U.S. provisional patent application Ser. No. 60/461,094, attorney docket no. 25791.272, filed on Apr. 8, 2003, (117) U.S. provisional patent application Ser. No. 60/461,038, attorney docket no. 25791.273, filed on Apr. 7, 2003, (118) U.S. provisional patent application Ser. No. 60/463,586, attorney docket no. 25791.277, filed on Apr. 17, 2003, (119) U.S. provisional patent application Ser. No. 60/472,240, attorney docket no. 25791.286, filed on May 20, 2003, (120) U.S. patent application Ser. No. 10/619,285, attorney docket no. 25791.292, filed on Jul. 14, 2003, which is a continuation-in-part of U.S. utility patent application Ser. No. 09/969,922, attorney docket no. 25791.69, filed on Oct. 3, 2001, which is a continuation-in-part application of U.S. Pat. No. 6,328,113, which was filed as U.S. patent application Ser. No. 09/440,338, attorney docket number 25791.9.02, filed on Nov. 15, 1999, which claims priority from provisional application 60/108,558, filed on Nov. 16, 1998, (121) U.S. utility patent application Ser. No. 10/418,688, attorney docket no. 25791.257, which was filed on Apr. 18, 2003, as a division of U.S. utility patent application Ser. No. 09/523,468, attorney docket no. 25791.11.02, filed on Mar. 10, 2000, which claims priority from provisional application 60/124,042, filed on Mar. 11, 1999, (122) PCT patent application Ser. No. PCT/US04/06246, attorney docket no. 25791.238.02, filed on Feb. 26, 2004, (123) PCT patent application serial number PCT/US04/08170, attorney docket number 25791.40.02, filed on Mar. 15/04, (124) PCT patent application serial number PCT/US04/08171, attorney docket number 25791.236.02, filed on Mar. 15, 2004, (125) PCT patent application serial number PCT/US04/08073, attorney docket number 25791.262.02, filed on Mar. 18, 2004, (126) PCT patent application serial number PCT/US04/07711, attorney docket number 25791.253.02, filed on Mar. 11, 2004, (127) PCT patent application serial number PCT/US2004/009434, attorney docket number 25791.260.02, filed on Mar. 26, 2004, (128) PCT patent application serial number PCT/US2004/010317, attorney docket number 25791.270.02, filed on Apr. 2, 2004, (129) PCT patent application serial number PCT/US2004/010712, attorney docket number 25791.272.02, filed on Apr. 6, 2004, (130) PCT patent application serial number PCT/US2004/010762, attorney docket number 25791.273.02, filed on Apr. 6, 2004, (131) PCT patent application serial number PCT/2004/011973, attorney docket number 25791.277.02, filed on Apr. 15, 2004, (132) U.S. provisional patent application Ser. No. 60/495,056, attorney docket number 25791.301, filed on Aug. 14, 2003, and (133) U.S. provisional patent application Ser. No. 60/585,370, attorney docket number 25791.299, filed on Jul. 2, 2004, the disclosures of which are incorporated herein by reference.

### BACKGROUND OF THE INVENTION

This invention relates generally to oil and gas exploration, and in particular to forming and repairing wellbore casings to facilitate oil and gas exploration.

### SUMMARY OF THE INVENTION

According to another aspect of the present invention, a method of determining the expandability of a selected tubular member is provided that includes determining an anisotropy value for the selected tubular member, determining a strain hardening value for the selected tubular member; and multiplying the anisotropy value times the strain hardening value to generate an expandability value for the selected tubular member.

According to another aspect of the present invention, a method of radially expanding and plastically deforming tubular members is provided that includes selecting a tubular member; determining an anisotropy value for the selected tubular member; determining a strain hardening value for the selected tubular member; multiplying the anisotropy value times the strain hardening value to generate an expandability value for the selected tubular member; and if the anisotropy value is greater than 0.12, then radially expanding and plastically deforming the selected tubular member.

According to another aspect of the present invention, a method of selecting tubular members for radial expansion and plastic deformation is provided that includes selecting a tubular member from a collection of tubular member; determining a carbon content of the selected tubular member; determining a carbon equivalent value for the selected tubular member; and if the carbon content of the selected tubular member is less than or equal to 0.12 percent and the carbon equivalent value for the selected tubular member is less than 0.21, then determining that the selected tubular member is suitable for radial expansion and plastic deformation.

According to another aspect of the present invention, a method of selecting tubular members for radial expansion and plastic deformation is provided that includes selecting a tubular member from a collection of tubular member; determining a carbon content of the selected tubular member; determining a carbon equivalent value for the selected tubular member; and if the carbon content of the selected tubular member is greater than 0.12 percent and the carbon equivalent value for the selected tubular member is less than 0.36, then determining that the selected tubular member is suitable for radial expansion and plastic deformation.

### BRIEF DESCRIPTION OF THE DRAWINGS

FIG. 1 is a fragmentary cross sectional view of an exemplary embodiment of an expandable tubular member positioned within a preexisting structure.

FIG. 2 is a fragmentary cross sectional view of the expandable tubular member of FIG. 1 after positioning an expansion device within the expandable tubular member.

FIG. 3 is a fragmentary cross sectional view of the expandable tubular member of FIG. 2 after operating the expansion device within the expandable tubular member to radially expand and plastically deform a portion of the expandable tubular member.

FIG. 4 is a fragmentary cross sectional view of the expandable tubular member of FIG. 3 after operating the expansion device within the expandable tubular member to radially expand and plastically deform another portion of the expandable tubular member.

FIG. 5 is a graphical illustration of exemplary embodiments of the stress/strain curves for several portions of the expandable tubular member of FIGS. 1-4.

FIG. 6 is a graphical illustration of the an exemplary embodiment of the yield strength vs. ductility curve for at least a portion of the expandable tubular member of FIGS. 1-4.

FIG. 7 is a fragmentary cross sectional illustration of an embodiment of a series of overlapping expandable tubular members.

FIG. 8 is a fragmentary cross sectional view of an exemplary embodiment of an expandable tubular member positioned within a preexisting structure.

FIG. 9 is a fragmentary cross sectional view of the expandable tubular member of FIG. 8 after positioning an expansion device within the expandable tubular member.

FIG. 10 is a fragmentary cross sectional view of the expandable tubular member of FIG. 9 after operating the expansion device within the expandable tubular member to radially expand and plastically deform a portion of the expandable tubular member.

FIG. 11 is a fragmentary cross sectional view of the expandable tubular member of FIG. 10 after operating the expansion device within the expandable tubular member to radially expand and plastically deform another portion of the expandable tubular member.

FIG. 12 is a graphical illustration of exemplary embodiments of the stress/strain curves for several portions of the expandable tubular member of FIGS. 8-11.

FIG. 13 is a graphical illustration of an exemplary embodiment of the yield strength vs. ductility curve for at least a portion of the expandable tubular member of FIGS. 8-11.

FIG. 14 is a fragmentary cross sectional view of an exemplary embodiment of an expandable tubular member positioned within a preexisting structure.

FIG. 15 is a fragmentary cross sectional view of the expandable tubular member of FIG. 14 after positioning an expansion device within the expandable tubular member.

FIG. 16 is a fragmentary cross sectional view of the expandable tubular member of FIG. 15 after operating the expansion device within the expandable tubular member to radially expand and plastically deform a portion of the expandable tubular member.

FIG. 17 is a fragmentary cross sectional view of the expandable tubular member of FIG. 16 after operating the expansion device within the expandable tubular member to radially expand and plastically deform another portion of the expandable tubular member.

FIG. 18 is a flow chart illustration of an exemplary embodiment of a method of processing an expandable tubular member.

FIG. 19 is a graphical illustration of the an exemplary embodiment of the yield strength vs. ductility curve for at least a portion of the expandable tubular member during the operation of the method of FIG. 18.

FIG. 20 is a graphical illustration of stress/strain curves for an exemplary embodiment of an expandable tubular member.

FIG. 21 is a graphical illustration of stress/strain curves for an exemplary embodiment of an expandable tubular member.

FIG. 35a is a fragmentary cross-sectional illustration of an exemplary embodiment of an expandable tubular member.

FIG. 35b is a graphical illustration of an exemplary embodiment of the variation in the yield point for the expandable tubular member of FIG. 35a.

FIG. 36a is a flow chart illustration of an exemplary embodiment of a method for processing a tubular member.

FIG. 36b is an illustration of the microstructure of an exemplary embodiment of a tubular member prior to thermal processing.

FIG. 36c is an illustration of the microstructure of an exemplary embodiment of a tubular member after thermal processing.

FIG. 37a is a flow chart illustration of an exemplary embodiment of a method for processing a tubular member.

FIG. 37b is an illustration of the microstructure of an exemplary embodiment of a tubular member prior to thermal processing.

FIG. 37c is an illustration of the microstructure of an exemplary embodiment of a tubular member after thermal processing.

FIG. 38a is a flow chart illustration of an exemplary embodiment of a method for processing a tubular member.

FIG. 38b is an illustration of the microstructure of an exemplary embodiment of a tubular member prior to thermal processing.

FIG. 38c is an illustration of the microstructure of an exemplary embodiment of a tubular member after thermal processing.

### DETAILED DESCRIPTION OF THE ILLUSTRATIVE EMBODIMENTS

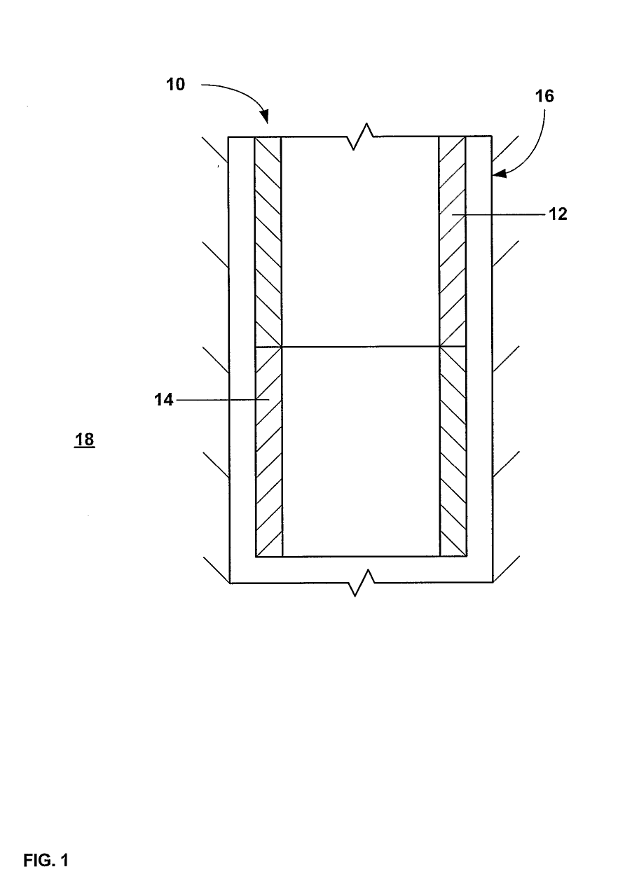

Referring initially to FIG. 1, an exemplary embodiment of an expandable tubular assembly 10 includes a first expandable tubular member 12 coupled to a second expandable tubular member 14. In several exemplary embodiments, the ends of the first and second expandable tubular members, 12 and 14, are coupled using, for example, a conventional mechanical coupling, a welded connection, a brazed connection, a threaded connection, and/or an interference fit connection. In an exemplary embodiment, the first expandable tubular member 12 has a plastic yield point YP₁, and the second expandable tubular member 14 has a plastic yield point YP₂. In an exemplary embodiment, the expandable tubular assembly 10 is positioned within a preexisting structure such as, for example, a wellbore 16 that traverses a subterranean formation 18.

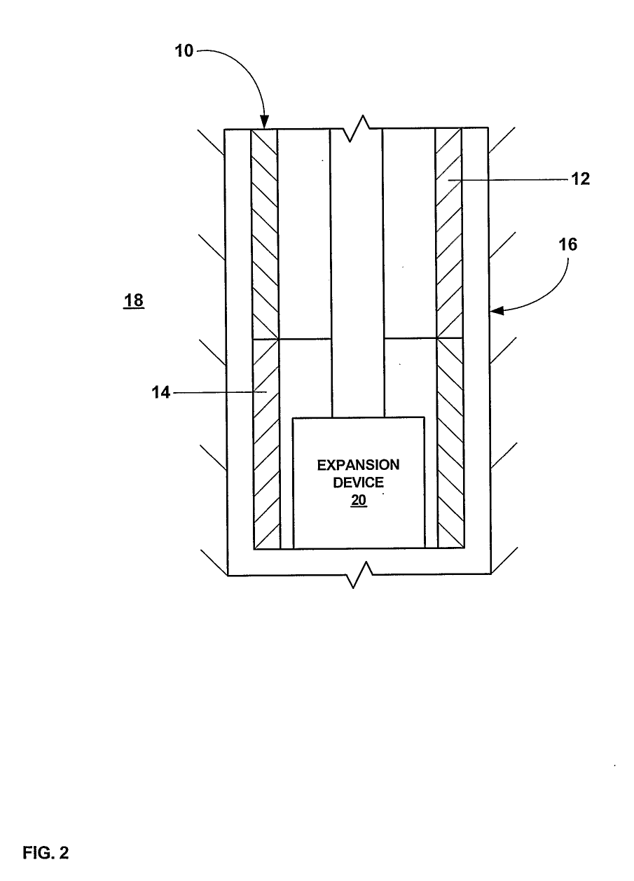

As illustrated in FIG. 2, an expansion device 20 may then be positioned within the second expandable tubular member 14. In several exemplary embodiments, the expansion device 20 may include, for example, one or more of the following conventional expansion devices: a) an expansion cone; b) a rotary expansion device; c) a hydroforming expansion device; d) an impulsive force expansion device; d) any one of the expansion devices commercially available from, or disclosed in any of the published patent applications or issued patents, of Weatherford International, Baker Hughes, Halliburton Energy Services, Shell Oil Co., Schlumberger, and/or Enventure Global Technology L.L.C. In several exemplary embodiments, the expansion device 20 is positioned within the second expandable tubular member 14 before, during, or after the placement of the expandable tubular assembly 10 within the preexisting structure 16.

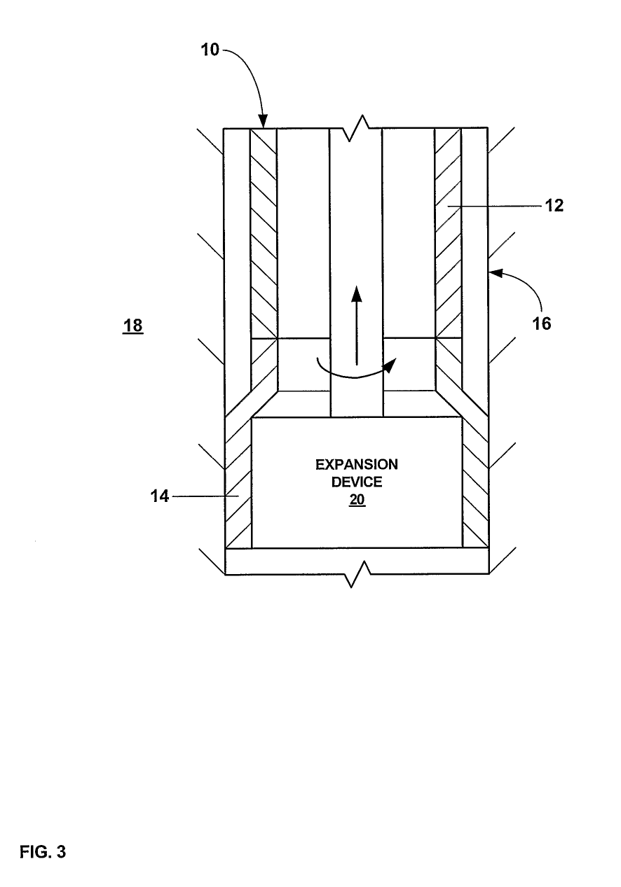

As illustrated in FIG. 3, the expansion device 20 may then be operated to radially expand and plastically deform at least a portion of the second expandable tubular member 14 to form a bell-shaped section.

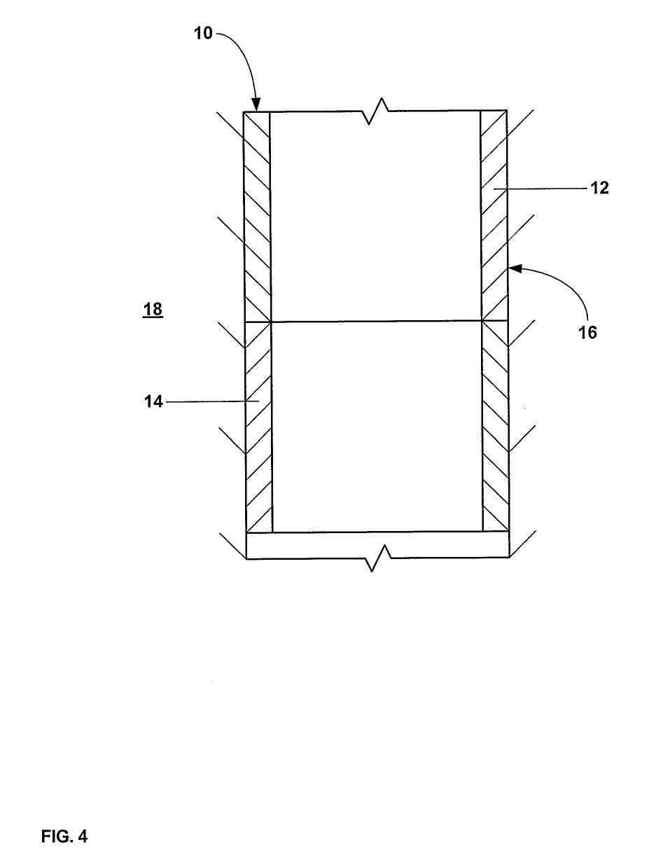

As illustrated in FIG. 4, the expansion device 20 may then be operated to radially expand and plastically deform the remaining portion of the second expandable tubular member 14 and at least a portion of the first expandable tubular member 12.

In an exemplary embodiment, at least a portion of at least a portion of at least one of the first and second expandable tubular members, 12 and 14, are radially expanded into intimate contact with the interior surface of the preexisting structure 16.

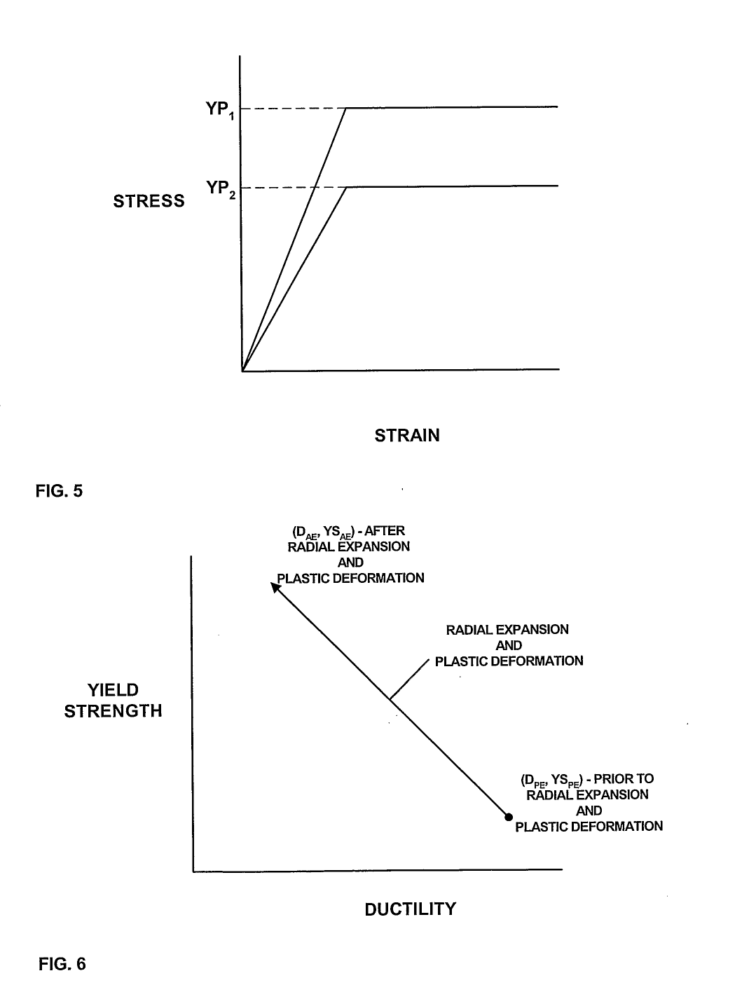

In an exemplary embodiment, as illustrated in FIG. 5, the plastic yield point YP₁ is greater than the plastic yield point YP₂. In this manner, in an exemplary embodiment, the amount of power and/or energy required to radially expand the second expandable tubular member 14 is less than the amount of power and/or energy required to radially expand the first expandable tubular member 12.

In an exemplary embodiment, as illustrated in FIG. 6, the first expandable tubular member 12 and/or the second expandable tubular member 14 have a ductility D_{PE} and a yield strength YS_{PE} prior to radial expansion and plastic deformation, and a ductility D_{AE} and a yield strength YS_{AE} after radial expansion and plastic deformation. In an exemplary embodiment, D_{PE} is greater than D_{AE}, and YS_{AE} is greater than YS_{PE}. In this manner, the first expandable tubular member 12 and/or the second expandable tubular member 14 are transformed during the radial expansion and plastic deformation process. Furthermore, in this manner, in an exemplary embodiment, the amount of power and/or energy required to radially expand each unit length of the first and/or second expandable tubular members, 12 and 14, is reduced. Furthermore, because the YS_{AE} is greater than YS_{PE}, the collapse strength of the first expandable tubular member 12 and/or the second expandable tubular member 14 is increased after the radial expansion and plastic deformation process.

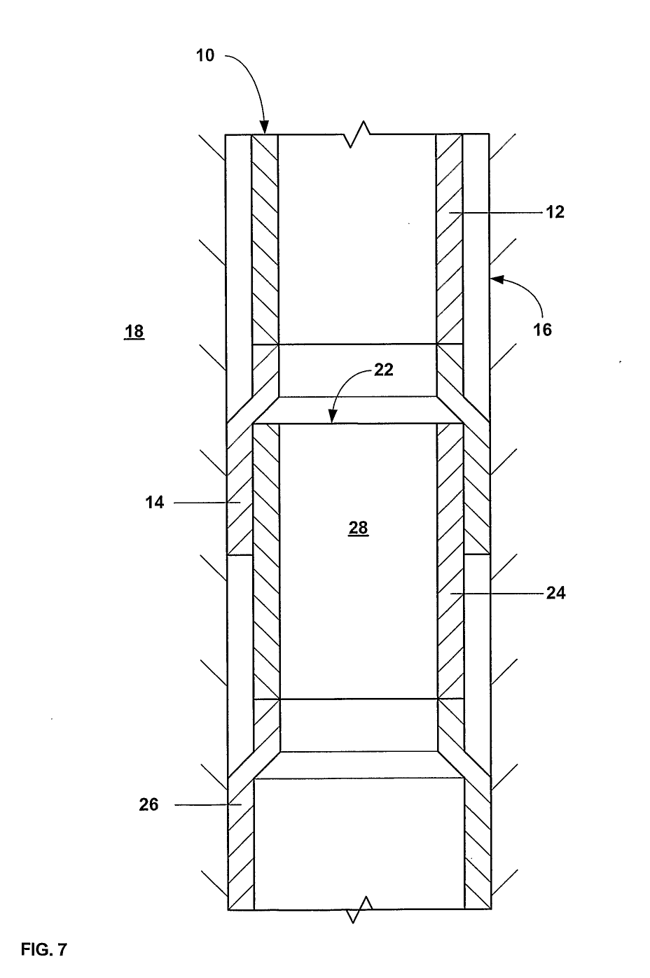

In an exemplary embodiment, as illustrated in FIG. 7, following the completion of the radial expansion and plastic deformation of the expandable tubular assembly 10 described above with reference to FIGS. 1-4, at least a portion of the second expandable tubular member 14 has an inside diameter that is greater than at least the inside diameter of the first expandable tubular member 12. In this manner a bell-shaped section is formed using at least a portion of the second expandable tubular member 14. Another expandable tubular assembly 22 that includes a first expandable tubular member 24 and a second expandable tubular member 26 may then be positioned in overlapping relation to the first expandable tubular assembly 10 and radially expanded and plastically deformed using the methods described above with reference to FIGS. 1-4. Furthermore, following the completion of the radial expansion and plastic deformation of the expandable tubular assembly 20, in an exemplary embodiment, at least a portion of the second expandable tubular member 26 has an inside diameter that is greater than at least the inside diameter of the first expandable tubular member 24. In this manner a bell-shaped section is formed using at least a portion of the second expandable tubular member 26. Furthermore, in this manner, a mono-diameter tubular assembly is formed that defines an internal passage 28 having a substantially constant cross-sectional area and/or inside diameter.

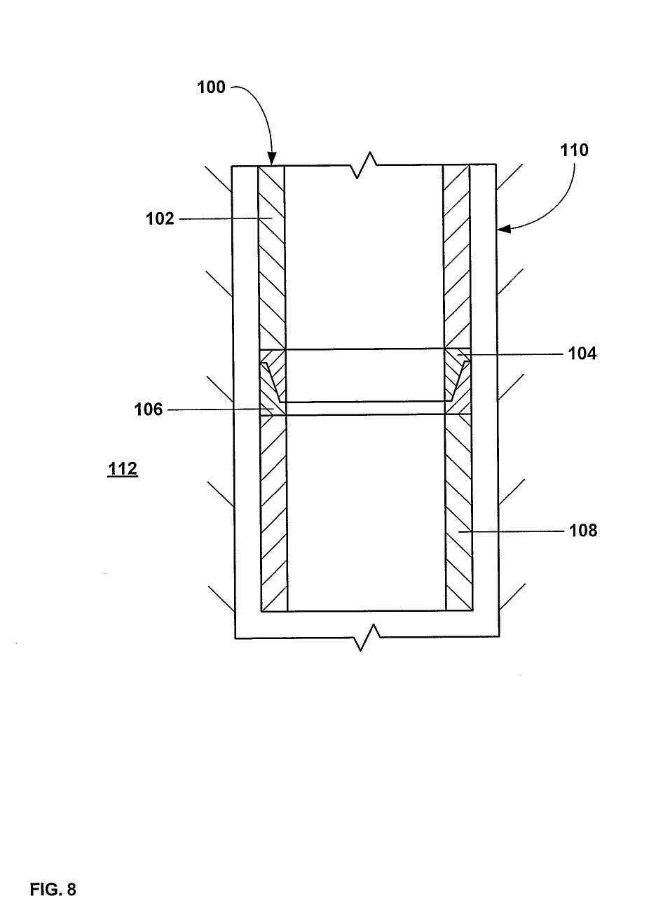

Referring to FIG. 8, an exemplary embodiment of an expandable tubular assembly 100 includes a first expandable tubular member 102 coupled to a tubular coupling 104. The tubular coupling 104 is coupled to a tubular coupling 106. The tubular coupling 106 is coupled to a second expandable tubular member 108. In several exemplary embodiments, the tubular couplings, 104 and 106, provide a tubular coupling assembly for coupling the first and second expandable tubular members, 102 and 108, together that may include, for example, a conventional mechanical coupling, a welded connection, a brazed connection, a threaded connection, and/or an interference fit connection. In an exemplary embodiment, the first and second expandable tubular members 12 have a plastic yield point YP₁, and the tubular couplings, 104 and 106, have a plastic yield point YP₂. In an exemplary embodiment, the expandable tubular assembly 100 is positioned within a preexisting structure such as, for example, a wellbore 110 that traverses a subterranean formation 112.

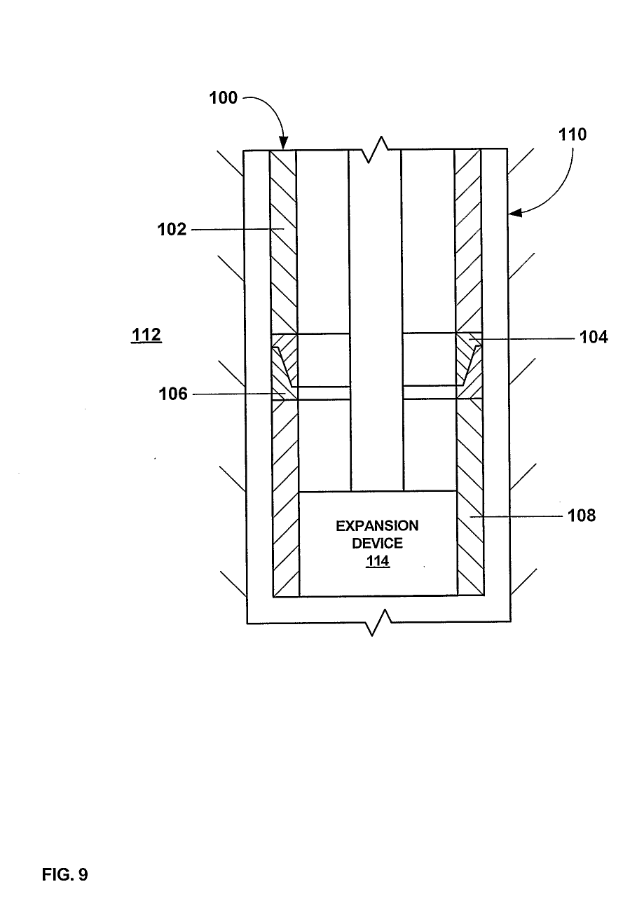

As illustrated in FIG. 9, an expansion device 114 may then be positioned within the second expandable tubular member 108. In several exemplary embodiments, the expansion device 114 may include, for example, one or more of the following conventional expansion devices: a) an expansion cone; b) a rotary expansion device; c) a hydroforming expansion device; d) an impulsive force expansion device; d) any one of the expansion devices commercially available from, or disclosed in any of the published patent applications or issued patents, of Weatherford International, Baker Hughes, Halliburton Energy Services, Shell Oil Co., Schlumberger, and/or Enventure Global Technology L.L.C. In several exemplary embodiments, the expansion device 114 is positioned within the second expandable tubular member 108 before, during, or after the placement of the expandable tubular assembly 100 within the preexisting structure 110.

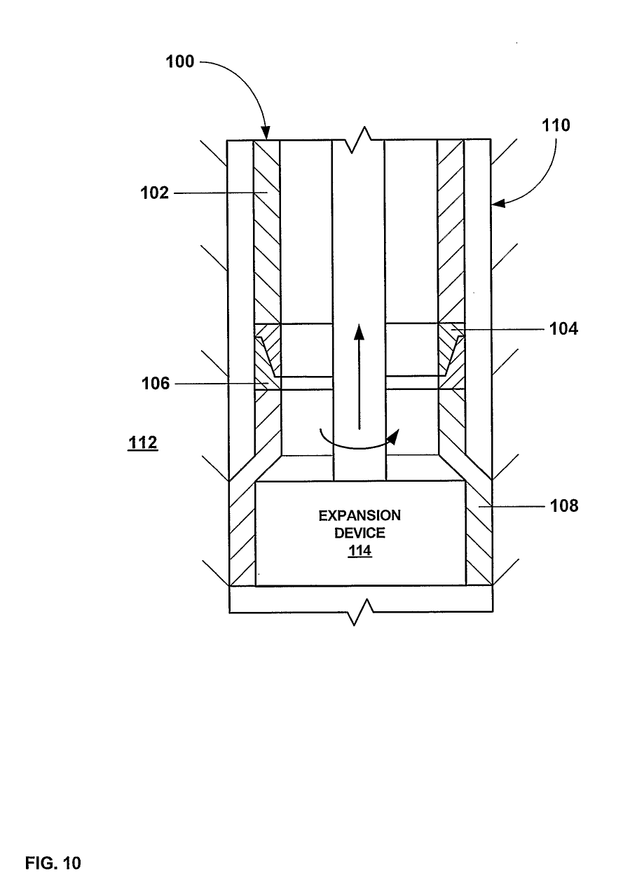

As illustrated in FIG. 10, the expansion device 114 may then be operated to radially expand and plastically deform at least a portion of the second expandable tubular member 108 to form a bell-shaped section.

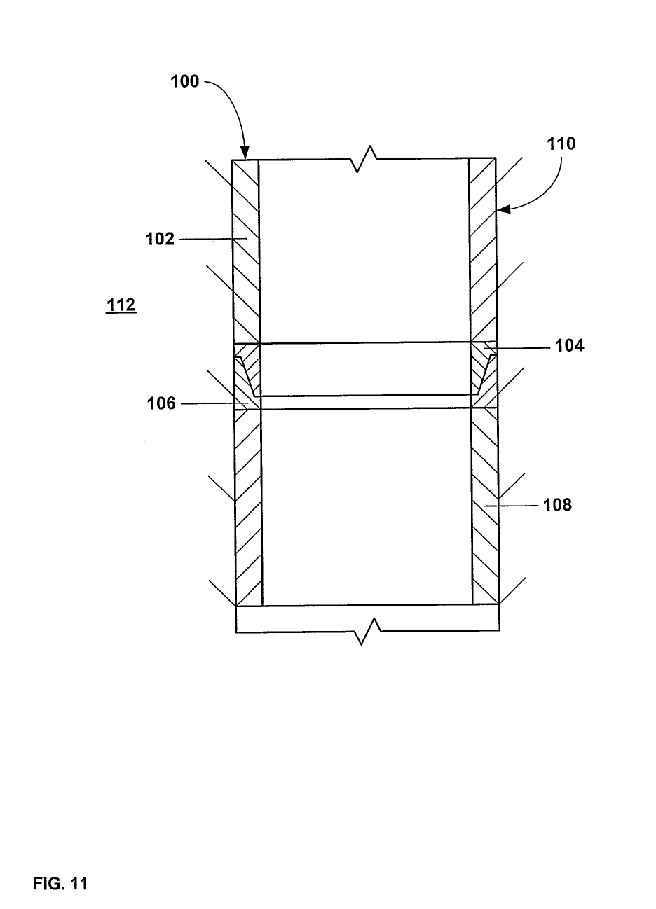

As illustrated in FIG. 11, the expansion device 114 may then be operated to radially expand and plastically deform the remaining portion of the second expandable tubular member 108, the tubular couplings, 104 and 106, and at least a portion of the first expandable tubular member 102.

In an exemplary embodiment, at least a portion of at least a portion of at least one of the first and second expandable tubular members, 102 and 108, are radially expanded into intimate contact with the interior surface of the preexisting structure 110.

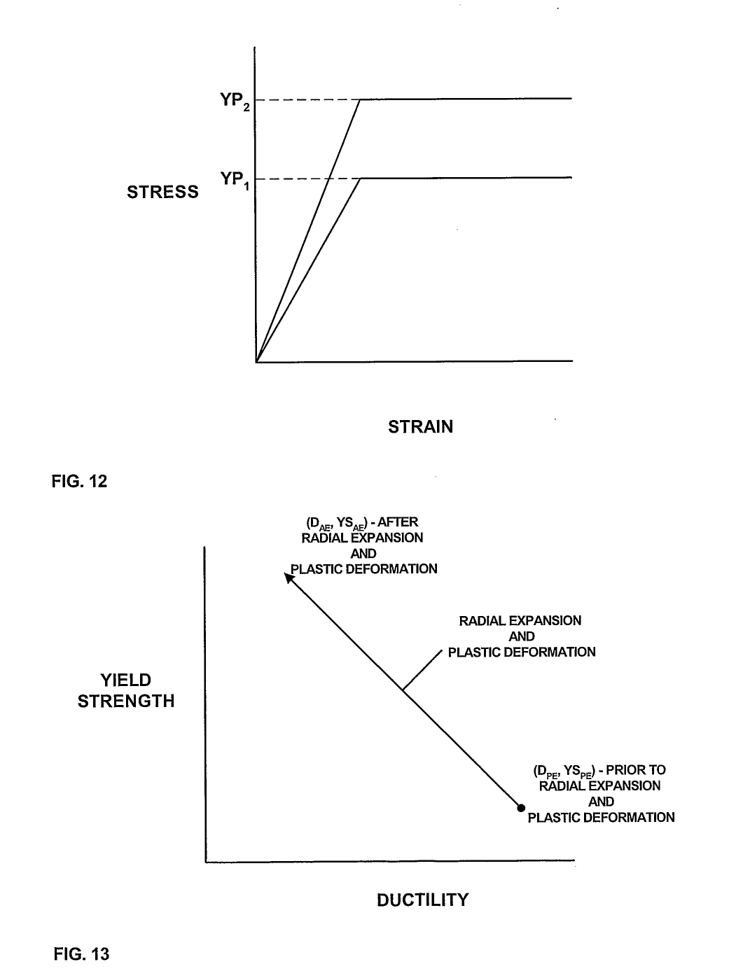

In an exemplary embodiment, as illustrated in FIG. 12, the plastic yield point YP₁ is less than the plastic yield point YP₂. In this manner, in an exemplary embodiment, the amount of power and/or energy required to radially expand each unit length of the first and second expandable tubular members, 102 and 108, is less than the amount of power and/or energy required to radially expand each unit length of the tubular couplings, 104 and 106.

In an exemplary embodiment, as illustrated in FIG. 13, the first expandable tubular member 12 and/or the second expandable tubular member 14 have a ductility D_{PE} and a yield strength YS_{PE} prior to radial expansion and plastic deformation, and a ductility D_{AE} and a yield strength YS_{AE} after radial expansion and plastic deformation. In an exemplary embodiment, D_{PE} is greater than D_{AE}, and YS_{AE} is greater than YS_{PE}. In this manner, the first expandable tubular member 12 and/or the second expandable tubular member 14 are transformed during the radial expansion and plastic deformation process. Furthermore, in this manner, in an exemplary embodiment, the amount of power and/or energy required to radially expand each unit length of the first and/or second expandable tubular members, 12 and 14, is reduced. Furthermore, because the YS_{AE} is greater than YS_{PE}, the collapse strength of the first expandable tubular member 12 and/or the second expandable tubular member 14 is increased after the radial expansion and plastic deformation process.

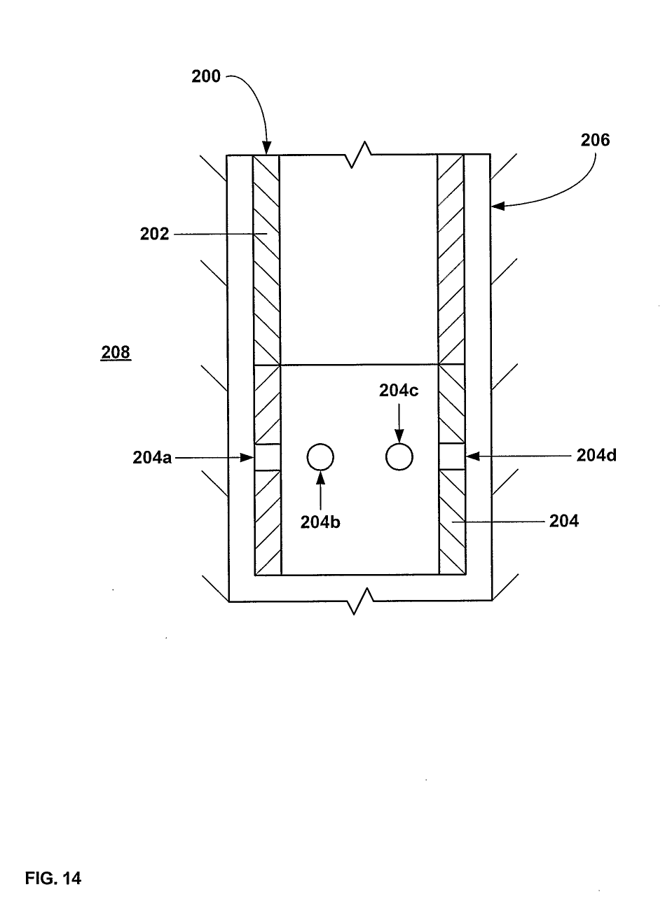

Referring to FIG. 14, an exemplary embodiment of an expandable tubular assembly 200 includes a first expandable tubular member 202 coupled to a second expandable tubular member 204 that defines radial openings 204a, 204b, 204c, and 204d. In several exemplary embodiments, the ends of the first and second expandable tubular members, 202 and 204, are coupled using, for example, a conventional mechanical coupling, a welded connection, a brazed connection, a threaded connection, and/or an interference fit connection. In an exemplary embodiment, one or more of the radial openings, 204a, 204b, 204c, and 204d, have circular, oval, square, and/or irregular cross sections and/or include portions that extend to and interrupt either end of the second expandable tubular member 204. In an exemplary embodiment, the expandable tubular assembly 200 is positioned within a preexisting structure such as, for example, a wellbore 206 that traverses a subterranean formation 208.

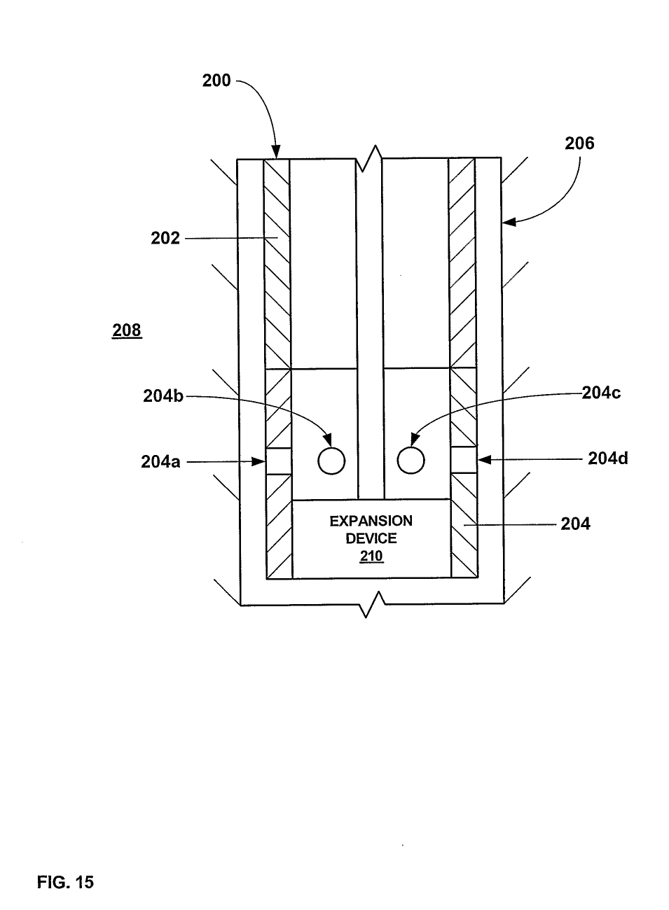

As illustrated in FIG. 15, an expansion device 210 may then be positioned within the second expandable tubular member 204. In several exemplary embodiments, the expansion device 210 may include, for example, one or more of the following conventional expansion devices: a) an expansion cone; b) a rotary expansion device; c) a hydroforming expansion device; d) an impulsive force expansion device; d) any one of the expansion devices commercially available from, or disclosed in any of the published patent applications or issued patents, of Weatherford International, Baker Hughes, Halliburton Energy Services, Shell Oil Co., Schlumberger, and/or Enventure Global Technology L.L.C. In several exemplary embodiments, the expansion device 210 is positioned within the second expandable tubular member 204 before, during, or after the placement of the expandable tubular assembly 200 within the preexisting structure 206.

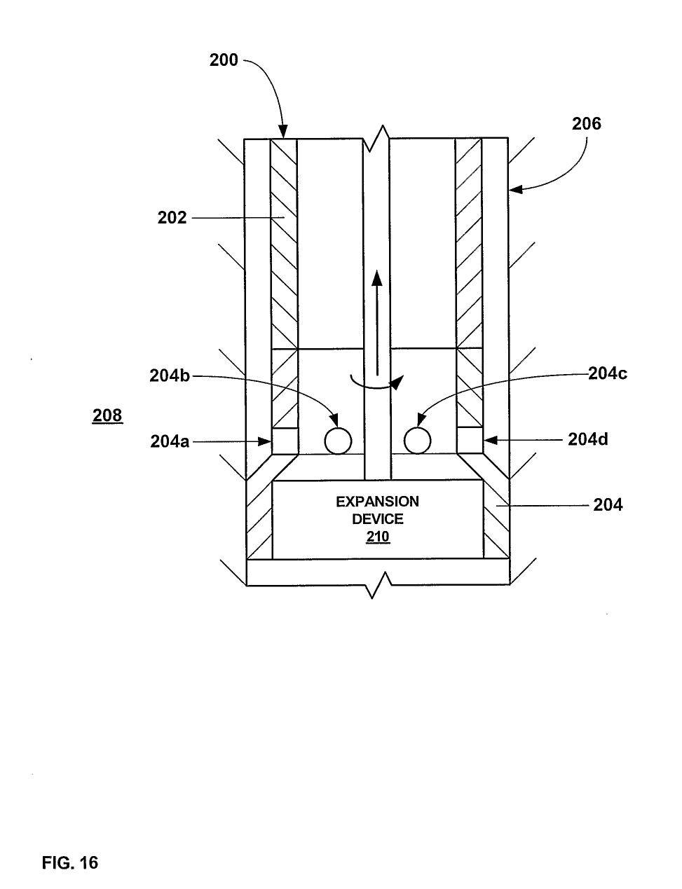

As illustrated in FIG. 16, the expansion device 210 may then be operated to radially expand and plastically deform at least a portion of the second expandable tubular member 204 to form a bell-shaped section.

As illustrated in FIG. 16, the expansion device 20 may then be operated to radially expand and plastically deform the remaining portion of the second expandable tubular member 204 and at least a portion of the first expandable tubular member 202.

In an exemplary embodiment, the anisotropy ratio AR for the first and second expandable tubular members is defined by the following equation:
AR=In(WT_{f}/WTₒ)/In(D_{f}/Dₒ);
where AR=anisotropy ratio;
where WT_{f}=final wall thickness of the expandable tubular member following the radial expansion and plastic deformation of the expandable tubular member;
where WTᵢ=initial wall thickness of the expandable tubular member prior to the radial expansion and plastic deformation of the expandable tubular member;
where D_{f}=final inside diameter of the expandable tubular member following the radial expansion and plastic deformation of the expandable tubular member; and
where Dᵢ=initial inside diameter of the expandable tubular member prior to the radial expansion and plastic deformation of the expandable tubular member.

In an exemplary embodiment, the anisotropy ratio AR for the first and/or second expandable tubular members, 204 and 204, is greater than 1.

In an exemplary experimental embodiment, the second expandable tubular member 204 had an anisotropy ratio AR greater than 1, and the radial expansion and plastic deformation of the second expandable tubular member did not result in any of the openings, 204a, 204b, 204c, and 204d, splitting or otherwise fracturing the remaining portions of the second expandable tubular member. This was an unexpected result.

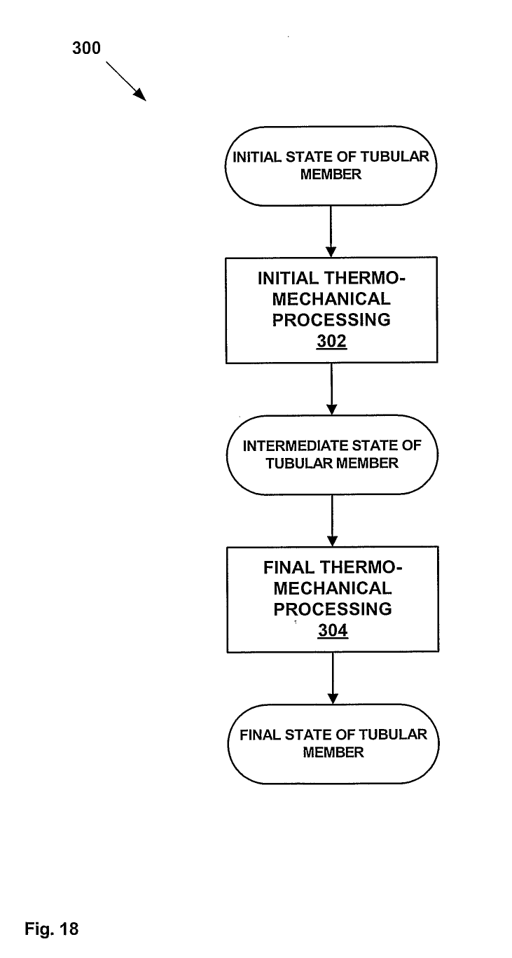

Referring to FIG. 18, in an exemplary embodiment, one or more of the expandable tubular members, 12, 14, 24, 26, 102, 104, 106, 108, 202 and/or 204 are processed using a method 300 in which a tubular member in an initial state is thermo-mechanically processed in step 302. In an exemplary embodiment, the thermo-mechanical processing 302 includes one or more heat treating and/or mechanical forming processes. As a result, of the thermo-mechanical processing 302, the tubular member is transformed to an intermediate state. The tubular member is then further thermo-mechanically processed in step 304. In an exemplary embodiment, the thermo-mechanical processing 304 includes one or more heat treating and/or mechanical forming processes. As a result, of the thermo-mechanical processing 304, the tubular member is transformed to a final state.

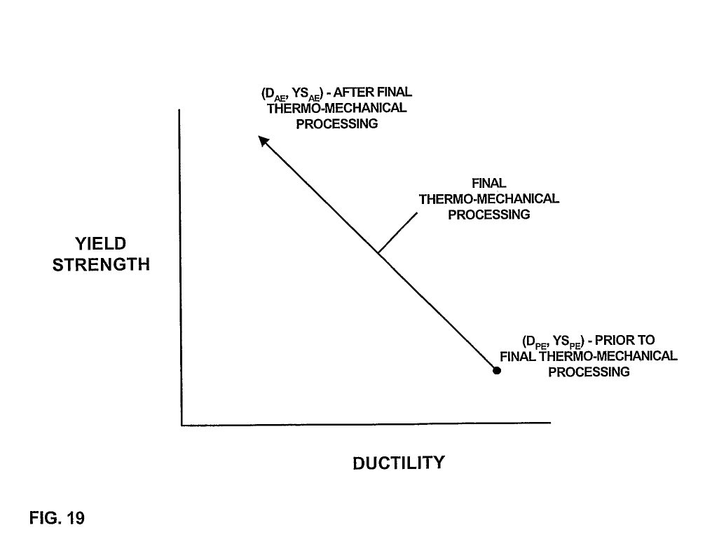

In an exemplary embodiment, as illustrated in FIG. 19, during the operation of the method 300, the tubular member has a ductility D_{PE} and a yield strength YS_{PE} prior to the final thermo-mechanical processing in step 304, and a ductility D_{AE} and a yield strength YS_{AE} after final thermo-mechanical processing. In an exemplary embodiment, D_{PE} is greater than D_{AE}, and YS_{AE} is greater than YS_{PE}. In this manner, the amount of energy and/or power required to transform the tubular member, using mechanical forming processes, during the final thermo-mechanical processing in step 304 is reduced. Furthermore, in this manner, because the YS_{AE} is greater than YS_{PE}, the collapse strength of the tubular member is increased after the final thermo-mechanical processing in step 304.

In an exemplary embodiment, one or more of the expandable tubular members, 12, 14, 24, 26, 102, 104, 106, 108, 202 and/or 204, have the following characteristics:

| Characteristic | Value |
|---|---|
| Tensile Strength | 60 to 120 ksi |
| Yield Strength | 50 to 100 ksi |
| Y/T Ratio | Maximum of 50/85% |
| Elongation During Radial Expansion and | Minimum of 35% |
| Plastic Deformation | |
| Width Reduction During Radial Expansion | Minimum of 40% |
| and Plastic Deformation | |
| Wall Thickness Reduction During Radial | Minimum of 30% |
| Expansion and Plastic Deformation | |
| Anisotropy | Minimum of 1.5 |
| Minimum Absorbed Energy at −4 F. (−20 C.) | 80 ft-lb |
| in the Longitudinal Direction | |
| Minimum Absorbed Energy at −4 F. (−20 C.) | 60 ft-lb |
| in the Transverse Direction | |
| Minimum Absorbed Energy at −4 F. (−20 C.) | 60 ft-lb |
| Transverse To A Weld Area | |
| Flare Expansion Testing | Minimum of 75% |
| | Without A Failure |
| Increase in Yield Strength Due To Radial | Greater than 5.4% |
| Expansion and Plastic Deformation | |

In an exemplary embodiment, one or more of the expandable tubular members, 12, 14, 24, 26, 102, 104, 106, 108, 202 and/or 204, are characterized by an expandability coefficient f:
i. f=r×n
ii. where f=expandability coefficient;
   1. r=anisotropy coefficient; and
   2. n=strain hardening exponent.

In an exemplary embodiment, the anisotropy coefficient for one or more of the expandable tubular members, 12, 14, 24, 26, 102, 104, 106, 108, 202 and/or 204 is greater than 1. In an exemplary embodiment, the strain hardening exponent for one or more of the expandable tubular members, 12, 14, 24, 26, 102, 104, 106, 108, 202 and/or 204 is greater than 0.12. In an exemplary embodiment, the expandability coefficient for one or more of the expandable tubular members, 12, 14, 24, 26, 102, 104, 106, 108, 202 and/or 204 is greater than 0.12.

In an exemplary embodiment, a tubular member having a higher expandability coefficient requires less power and/or energy to radially expand and plastically deform each unit length than a tubular member having a lower expandability coefficient. In an exemplary embodiment, a tubular member having a higher expandability coefficient requires less power and/or energy per unit length to radially expand and plastically deform than a tubular member having a lower expandability coefficient.

In several exemplary experimental embodiments, one or more of the expandable tubular members, 12, 14, 24, 26, 102, 104, 106, 108, 202 and/or 204, are steel alloys having one of the following compositions:

| Steel | Element and Percentage By Weight | | | | | | | |
|---|---|---|---|---|---|---|---|---|
| Alloy | C | Mn | P | S | Si | Cu | Ni | Cr |
| A | 0.065 | 1.44 | 0.01 | 0.002 | 0.24 | 0.01 | 0.01 | 0.02 |
| B | 0.18 | 1.28 | 0.017 | 0.004 | 0.29 | 0.01 | 0.01 | 0.03 |
| C | 0.08 | 0.82 | 0.006 | 0.003 | 0.30 | 0.16 | 0.05 | 0.05 |
| D | 0.02 | 1.31 | 0.02 | 0.001 | 0.45 | — | 9.1 | 18.7 |

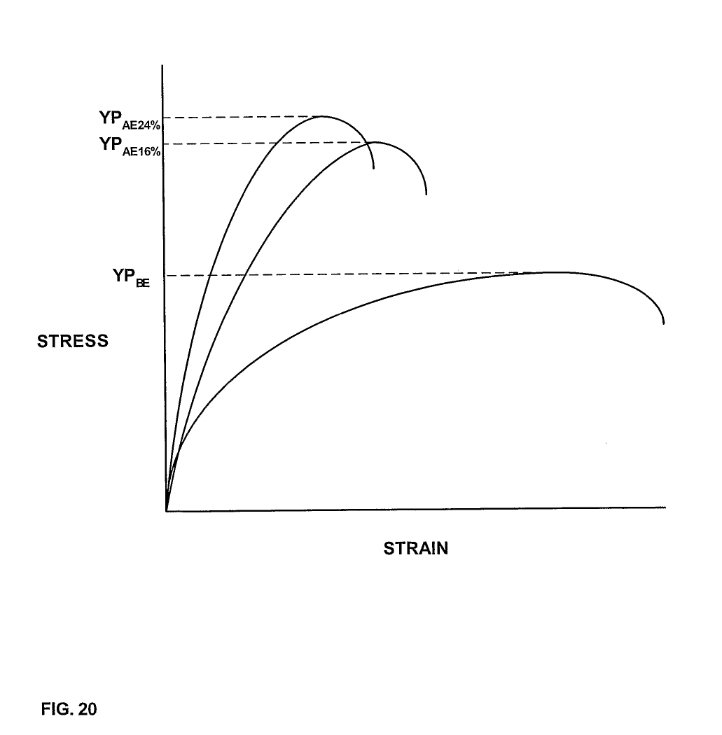

In exemplary experimental embodiment, as illustrated in FIG. 20, a sample of an expandable tubular member composed of Alloy A exhibited a yield point before radial expansion and plastic deformation YP_{BE}, a yield point after radial expansion and plastic deformation of about 16% YP_{AE16%}, and a yield point after radial expansion and plastic deformation of about 24% YP_{AE24%}. In an exemplary experimental embodiment, YP_{AE24%}>YP_{AE16%}>YP_{BE}. Furthermore, in an exemplary experimental embodiment, the ductility of the sample of the expandable tubular member composed of Alloy A also exhibited a higher ductility prior to radial expansion and plastic deformation than after radial expansion and plastic deformation. These were unexpected results.

In an exemplary experimental embodiment, a sample of an expandable tubular member composed of Alloy A exhibited the following tensile characteristics before and after radial expansion and plastic deformation:

| | | | | Width | Wall | |
|---|---|---|---|---|---|---|
| | Yield | | Elon- | Reduc- | Thickness | |
| | Point | Yield | gation | tion | Reduction | Anisot- |
| | ksi | Ratio | % | % | % | ropy |
| Before | 46.9 | 0.69 | 53 | −52 | 55 | 0.93 |
| Radial | | | | | | |
| Expansion | | | | | | |
| and Plastic | | | | | | |
| Deformation | | | | | | |
| After 16% | 65.9 | 0.83 | 17 | 42 | 51 | 0.78 |
| Radial | | | | | | |
| Expansion | | | | | | |
| After 24% | 68.5 | 0.83 | 5 | 44 | 54 | 0.76 |
| Radial | | | | | | |
| Expansion | | | | | | |
| % Increase | 40% for | | | | | |
| | 16% radial | | | | | |
| | expansion | | | | | |
| | 46% for | | | | | |
| | 24% radial | | | | | |
| | expansion | | | | | |

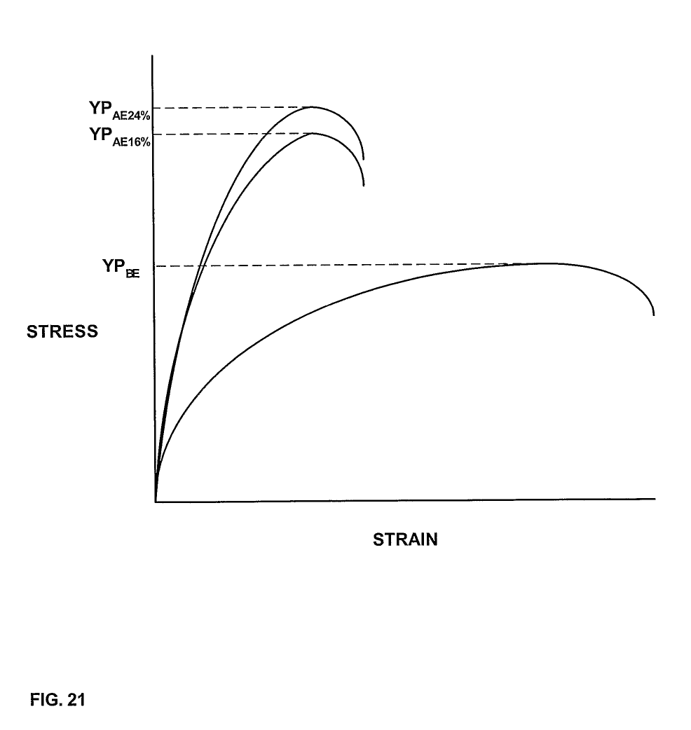

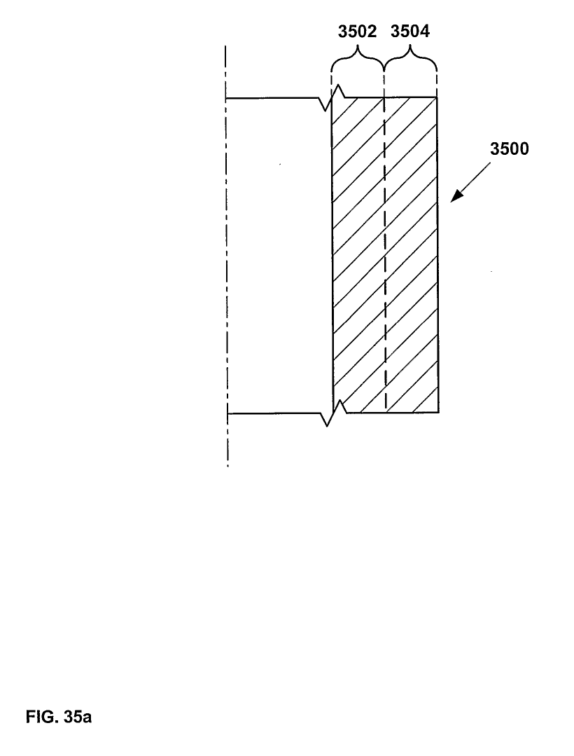

In exemplary experimental embodiment, as illustrated in FIG. 21, a sample of an expandable tubular member composed of Alloy B exhibited a yield point before radial expansion and plastic deformation YP_{BE}, a yield point after radial expansion and plastic deformation of about 16% YP_{AE16%}, and a yield point after radial expansion and plastic deformation of about 24% YP_{AE24%}. In an exemplary embodiment, YP_{AE24%}>YP_{AE16%}>YP_{BE}. Furthermore, in an exemplary experimental embodiment, the ductility of the sample of the expandable tubular member composed of Alloy B also exhibited a higher ductility prior to radial expansion and plastic deformation than after radial expansion and plastic deformation. These were unexpected results.

In an exemplary experimental embodiment, a sample of an expandable tubular member composed of Alloy B exhibited the following tensile characteristics before and after radial expansion and plastic deformation:

| | | | | Width | Wall | |
|---|---|---|---|---|---|---|
| | Yield | | Elon- | Reduc- | Thickness | |
| | Point | Yield | gation | tion | Reduction | Anisot- |
| | ksi | Ratio | % | % | % | ropy |
| Before | 57.8 | 0.71 | 44 | 43 | 46 | 0.93 |
| Radial | | | | | | |
| Expansion | | | | | | |
| and Plastic | | | | | | |
| Deformation | | | | | | |
| After 16% | 74.4 | 0.84 | 16 | 38 | 42 | 0.87 |
| Radial | | | | | | |
| Expansion | | | | | | |
| After 24% | 79.8 | 0.86 | 20 | 36 | 42 | 0.81 |
| Radial | | | | | | |
| Expansion | | | | | | |
| % Increase | 28.7% | | | | | |
| | increase | | | | | |
| | for 16% | | | | | |
| | radial | | | | | |
| | expansion | | | | | |
| | 38% | | | | | |
| | increase | | | | | |
| | for 24% | | | | | |
| | radial | | | | | |
| | expansion | | | | | |

In an exemplary experimental embodiment, samples of expandable tubulars composed of Alloys A, B, C, and D exhibited the following tensile characteristics prior to radial expansion and plastic deformation:

| | | | Elon- | | Absorbed | |
|---|---|---|---|---|---|---|
| Steel | Yield | Yield | gation | Anisot- | Energy | Expandability |
| Alloy | ksi | Ratio | % | ropy | ft-lb | Coefficient |
| A | 47.6 | 0.71 | 44 | 1.48 | 145 | |
| B | 57.8 | 0.71 | 44 | 1.04 | 62.2 | |
| C | 61.7 | 0.80 | 39 | 1.92 | 268 | |
| D | 48 | 0.55 | 56 | 1.34 | — | |

In an exemplary embodiment, one or more of the expandable tubular members, 12, 14, 24, 26, 102, 104, 106, 108, 202 and/or 204 have a strain hardening exponent greater than 0.12, and a yield ratio is less than 0.85.

In an exemplary embodiment, the carbon equivalent Cₑ, for tubular members having a carbon content (by weight percentage) less than or equal to 0.12%, is given by the following expression:
Cₑ=C+Mn/6+(Cr+Mo+V+Ti+Nb)/5+(Ni+Cu)/15
where Cₑ=carbon equivalent value;
a. C=carbon percentage by weight;
b. Mn=manganese percentage by weight;
c. Cr=chromium percentage by weight;
d. Mo=molybdenum percentage by weight;
e. V=vanadium percentage by weight;
f. Ti=titanium percentage by weight;
g. Nb=niobium percentage by weight;
h. Ni=nickel percentage by weight; and
i. Cu=copper percentage by weight.

In an exemplary embodiment, the carbon equivalent value Cₑ, for tubular members having a carbon content less than or equal to 0.12% (by weight), for one or more of the expandable tubular members, 12, 14, 24, 26, 102, 104, 106, 108, 202 and/or 204 is less than 0.21.

In an exemplary embodiment, the carbon equivalent Cₑ, for tubular members having more than 0.12% carbon content (by weight), is given by the following expression:
Cₑ=C+Si/30+(Mn+Cu+Cr)/20+Ni/60+Mo/15+V/10+5*B
where Cₑ=carbon equivalent value;
a. C=carbon percentage by weight;
b. Si=silicon percentage by weight;
c. Mn=manganese percentage by weight;
d. Cu=copper percentage by weight;
e. Cr=chromium percentage by weight;
f. Ni=nickel percentage by weight;
g. Mo=molybdenum percentage by weight;
h. V=vanadium percentage by weight; and
i. B=boron percentage by weight.

In an exemplary embodiment, the carbon equivalent value Cₑ, for tubular members having greater than 0.12% carbon content (by weight), for one or more of the expandable tubular members, 12, 14, 24, 26, 102, 104, 106, 108, 202 and/or 204 is less than 0.36.

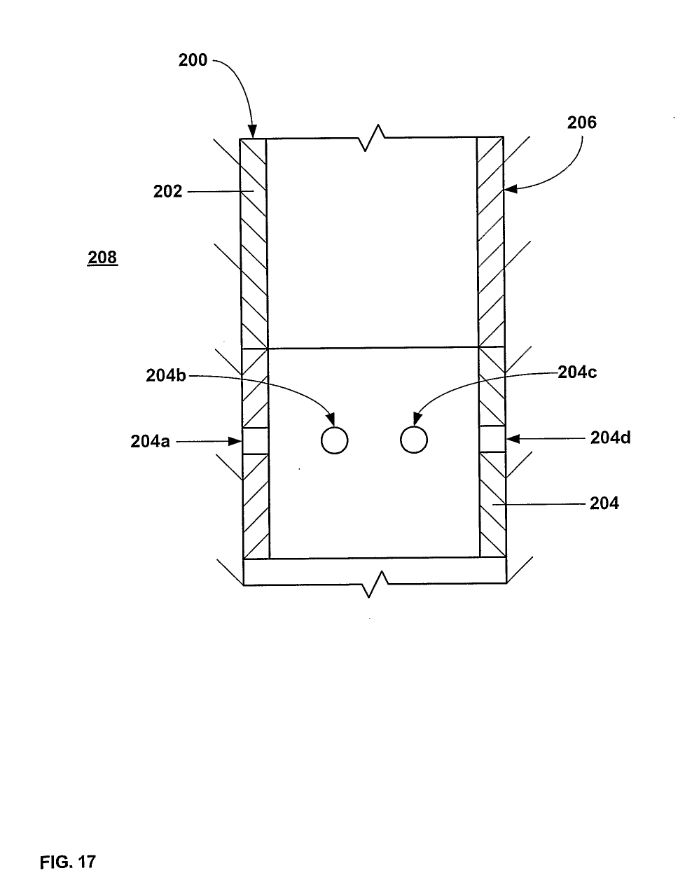

In several exemplary embodiments, the first and second tubular members described above with reference to FIGS. 1 to 21 are radially expanded and plastically deformed using the expansion device in a conventional manner and/or using one or more of the methods and apparatus disclosed in one or more of the following: The present application is related to the following: (1) U.S. patent application Ser. No. 09/454,139, attorney docket no. 25791.03.02, filed on Dec. 3, 1999, (2) U.S. patent application Ser. No. 09/510,913, attorney docket no. 25791.7.02, filed on Feb. 23, 2000, (3) U.S. patent application Ser. No. 09/502,350, attorney docket no. 25791.8.02, filed on Feb. 10, 2000, (4) U.S. patent application Ser. No. 09/440,338, attorney docket no. 25791.9.02, filed on Nov. 15, 1999, (5) U.S. patent application Ser. No. 09/523,460, attorney docket no. 25791.11.02, filed on Mar. 10, 2000, (6) U.S. patent application Ser. No. 09/512,895, attorney docket no. 25791.12.02, filed on Feb. 24, 2000, (7) U.S. patent application Ser. No. 09/511,941, attorney docket no. 25791.16.02, filed on Feb. 24, 2000, (8) U.S. patent application Ser. No. 09/588,946, attorney docket no. 25791.17.02, filed on Jun. 7, 2000, (9) U.S. patent application Ser. No. 09/559,122, attorney docket no. 25791.23.02, filed on Apr. 26, 2000, (10) PCT patent application serial no. PCT/US00/18635, attorney docket no. 25791.25.02, filed on Jul. 9, 2000, (11) U.S. provisional patent application Ser. No. 60/162,671, attorney docket no. 25791.27, filed on Nov. 1, 1999, (12) U.S. provisional patent application Ser. No. 60/154,047, attorney docket no. 25791.29, filed on Sep. 16, 1999, (13) U.S. provisional patent application Ser. No. 60/159,082, attorney docket no. 25791.34, filed on Oct. 12, 1999, (14) U.S. provisional patent application Ser. No. 60/159,039, attorney docket no. 25791.36, filed on Oct. 12, 1999, (15) U.S. provisional patent application Ser. No. 60/159,033, attorney docket no. 25791.37, filed on Oct. 12, 1999, (16) U.S. provisional patent application Ser. No. 60/212,359, attorney docket no. 25791.38, filed on Jun. 19, 2000, (17) U.S. provisional patent application Ser. No. 60/165,228, attorney docket no. 25791.39, filed on Nov. 12, 1999, (18) U.S. provisional patent application Ser. No. 60/221,443, attorney docket no. 25791.45, filed on Jul. 28, 2000, (19) U.S. provisional patent application Ser. No. 60/221,645, attorney docket no. 25791.46, filed on Jul. 28, 2000, (20) U.S. provisional patent application Ser. No. 60/233,638, attorney docket no. 25791.47, filed on Sep. 18, 2000, (21) U.S. provisional patent application Ser. No. 60/237,334, attorney docket no. 25791.48, filed on Oct. 2, 2000, (22) U.S. provisional patent application Ser. No. 60/270,007, attorney docket no. 25791.50, filed on Feb. 20, 2001, (23) U.S. provisional patent application Ser. No. 60/262,434, attorney docket no. 25791.51, filed on Jan. 17, 2001, (24) U.S. provisional patent application Ser. No. 60/259,486, attorney docket no. 25791.52, filed on Jan. 3, 2001, (25) U.S. provisional patent application Ser. No. 60/303,740, attorney docket no. 25791.61, filed on Jul. 6, 2001, (26) U.S. provisional patent application Ser. No. 60/313,453, attorney docket no. 25791.59, filed on Aug. 20, 2001, (27) U.S. provisional patent application Ser. No. 60/317,985, attorney docket no. 25791.67, filed on Sep. 6, 2001, (28) U.S. provisional patent application Ser. No. 60/3318,386, attorney docket no. 25791.67.02, filed on Sep. 10, 2001, (29) U.S. utility patent application Ser. No. 09/969,922, attorney docket no. 25791.69, filed on Oct. 3, 2001, (30) U.S. utility patent application Ser. No. 10/016,467, attorney docket no. 25791.70, filed on Dec. 10, 2001, (31) U.S. provisional patent application Ser. No. 60/343,674, attorney docket no. 25791.68, filed on Dec. 27, 2001; and (32) U.S. provisional patent application Ser. No. 60/346,309, attorney docket no. 25791.92, filed on Jan. 7, 2002, the disclosures of which are incorporated herein by reference.

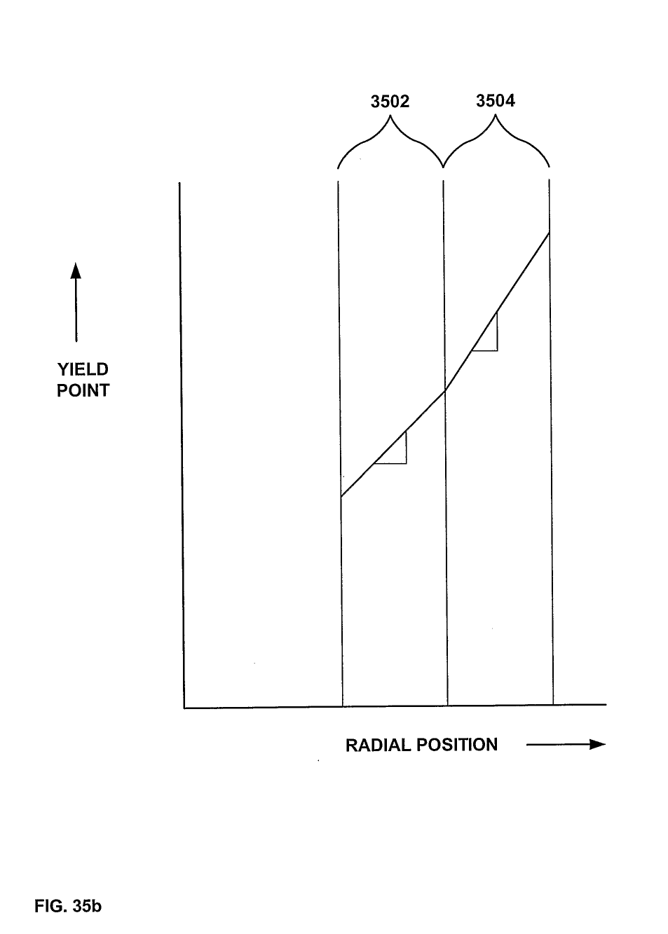

Referring to FIG. 35a an exemplary embodiment of an expandable tubular member 3500 includes a first tubular region 3502 and a second tubular portion 3504. In an exemplary embodiment, the material properties of the first and second tubular regions, 3502 and 3504, are different. In an exemplary embodiment, the yield points of the first and second tubular regions, 3502 and 3504, are different. In an exemplary embodiment, the yield point of the first tubular region 3502 is less than the yield point of the second tubular region 3504. In several exemplary embodiments, one or more of the expandable tubular members, 12, 14, 24, 26, 102, 104, 106, 108, 202 and/or 204 incorporate the tubular member 3500.

Referring to FIG. 35b, in an exemplary embodiment, the yield point within the first and second tubular regions, 3502a and 3502b, of the expandable tubular member 3502 vary as a function of the radial position within the expandable tubular member. In an exemplary embodiment, the yield point increases as a function of the radial position within the expandable tubular member 3502. In an exemplary embodiment, the relationship between the yield point and the radial position within the expandable tubular member 3502 is a linear relationship. In an exemplary embodiment, the relationship between the yield point and the radial position within the expandable tubular member 3502 is a non-linear relationship. In an exemplary embodiment, the yield point increases at different rates within the first and second tubular regions, 3502a and 3502b, as a function of the radial position within the expandable tubular member 3502. In an exemplary embodiment, the functional relationship, and value, of the yield points within the first and second tubular regions, 3502a and 3502b, of the expandable tubular member 3502 are modified by the radial expansion and plastic deformation of the expandable tubular member.

In several exemplary embodiments, one or more of the expandable tubular members, 12, 14, 24, 26, 102, 104, 106, 108, 202, 204 and/or 3502, prior to a radial expansion and plastic deformation, include a microstructure that is a combination of a hard phase, such as martensite, a soft phase, such as ferrite, and a transitionary phase, such as retained austentite. In this manner, the hard phase provides high strength, the soft phase provides ductility, and the transitionary phase transitions to a hard phase, such as martensite, during a radial expansion and plastic deformation. Furthermore, in this manner, the yield point of the tubular member increases as a result of the radial expansion and plastic deformation. Further, in this manner, the tubular member is ductile, prior to the radial expansion and plastic deformation, thereby facilitating the radial expansion and plastic deformation. In an exemplary embodiment, the composition of a dual-phase expandable tubular member includes (weight percentages): about 0.1% C, 1.2% Mn, and 0.3% Si.

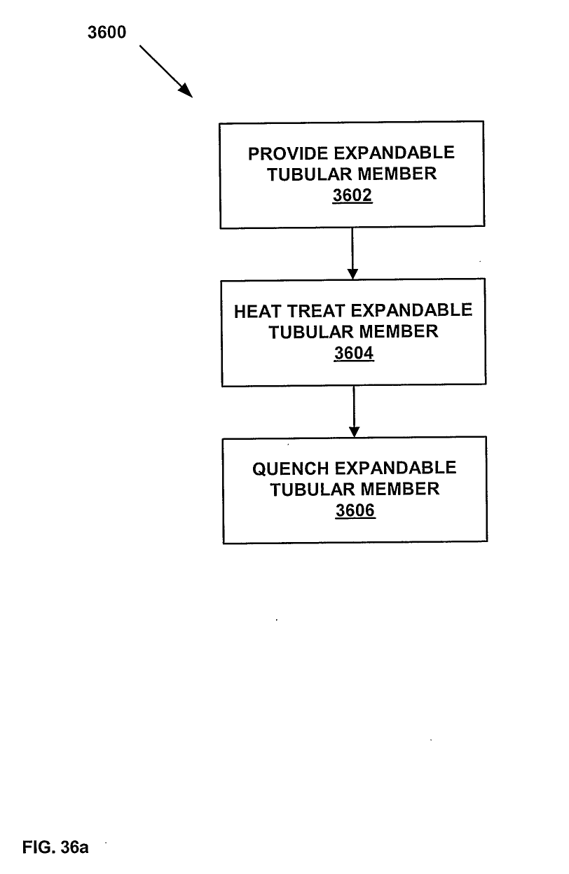

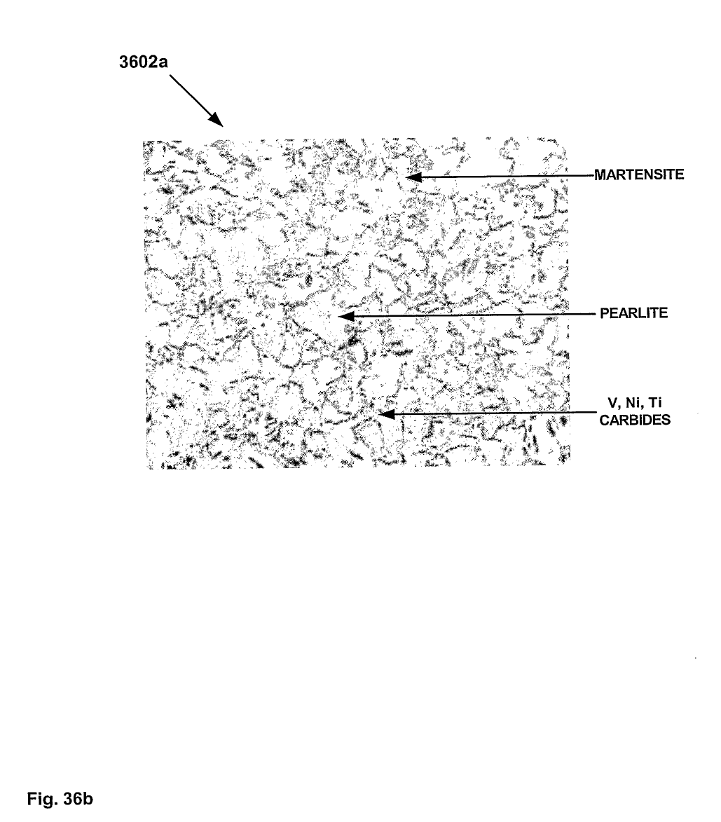

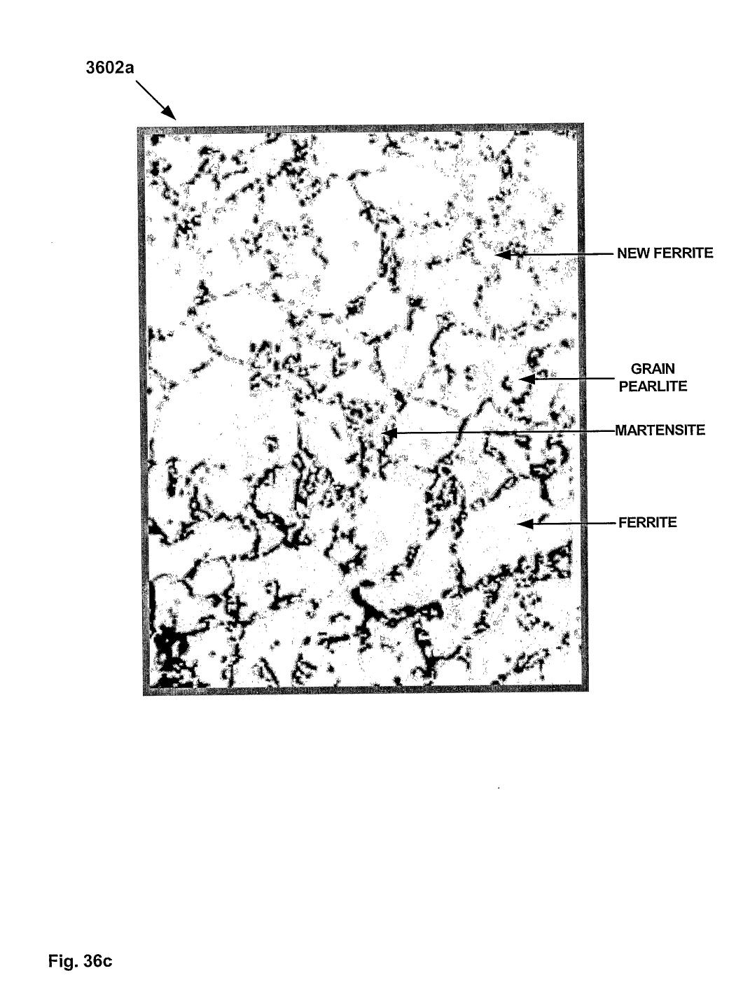

In an exemplary experimental embodiment, as illustrated in FIGS. 36a-36c, one or more of the expandable tubular members, 12, 14, 24, 26, 102, 104, 106, 108, 202, 204 and/or 3502 are processed in accordance with a method 3600, in which, in step 3602, an expandable tubular member 3602a is provided that is a steel alloy having following material composition (by weight percentage): 0.065% C, 1.44% Mn, 0.01% P, 0.002% S, 0.24% Si, 0.01% Cu, 0.01% Ni, 0.02% Cr, 0.05% V, 0.01% Mo, 0.01% Nb, and 0.01% Ti. In an exemplary experimental embodiment, the expandable tubular member 3602a provided in step 3602 has a yield strength of 45 ksi, and a tensile strength of 69 ksi.

In an exemplary experimental embodiment, as illustrated in FIG. 36b, in step 3602, the expandable tubular member 3602a includes a microstructure that includes martensite, pearlite, and V, Ni, and/or Ti carbides.

In an exemplary embodiment, the expandable tubular member 3602a is then heated at a temperature of 790° C. for about 10 minutes in step 3604.

In an exemplary embodiment, the expandable tubular member 3602a is then quenched in water in step 3606.

In an exemplary experimental embodiment, as illustrated in FIG. 36c, following the completion of step 3606, the expandable tubular member 3602a includes a microstructure that includes new ferrite, grain pearlite, martensite, and ferrite. In an exemplary experimental embodiment, following the completion of step 3606, the expandable tubular member 3602a has a yield strength of 67 ksi, and a tensile strength of 95 ksi.

In an exemplary embodiment, the expandable tubular member 3602a is then radially expanded and plastically deformed using one or more of the methods and apparatus described above. In an exemplary embodiment, following the radial expansion and plastic deformation of the expandable tubular member 3602a, the yield strength of the expandable tubular member is about 95 ksi.

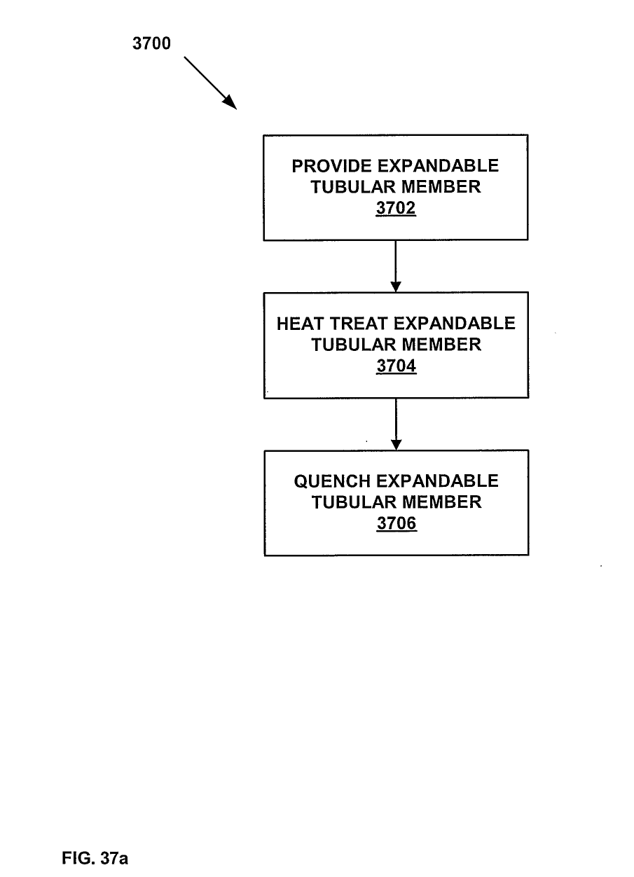

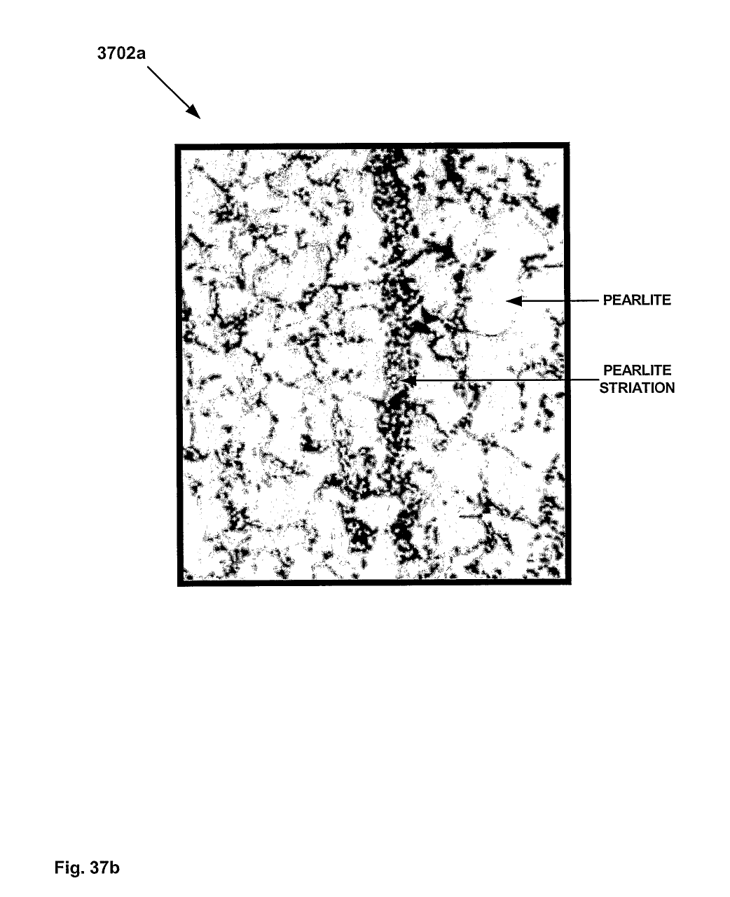

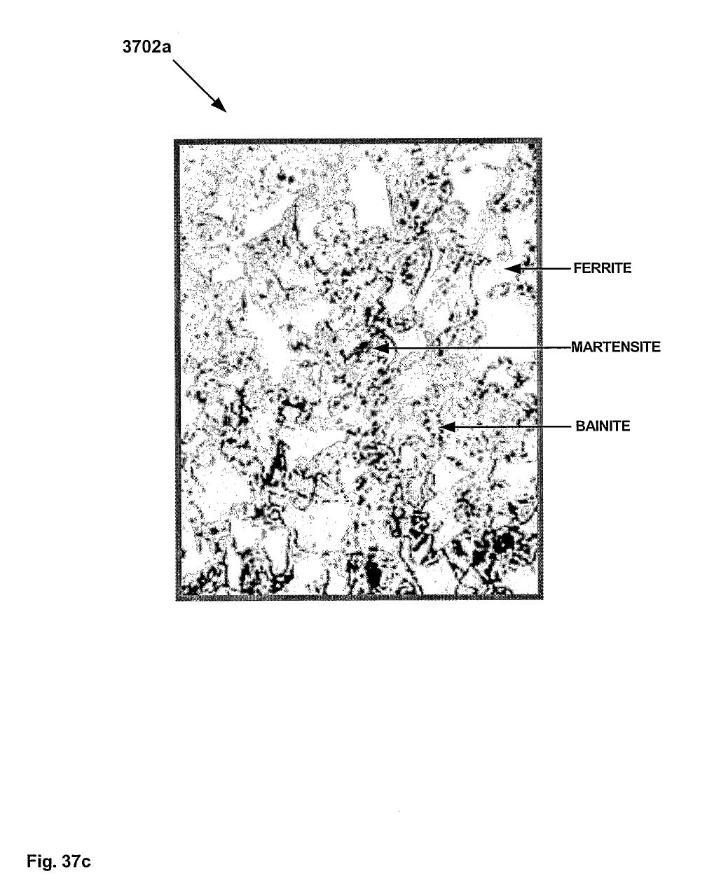

In an exemplary experimental embodiment, as illustrated in FIGS. 37a-37c, one or more of the expandable tubular members, 12, 14, 24, 26, 102, 104, 106, 108, 202, 204 and/or 3502 are processed in accordance with a method 3700, in which, in step 3702, an expandable tubular member 3702a is provided that is a steel alloy having following material composition (by weight percentage): 0.18% C, 1.28% Mn, 0.017% P, 0.004% S, 0.29% Si, 0.01% Cu, 0.01% Ni, 0.03% Cr, 0.04% V, 0.01% Mo, 0.03% Nb, and 0.01% Ti. In an exemplary experimental embodiment, the expandable tubular member 3702a provided in step 3702 has a yield strength of 60 ksi, and a tensile strength of 80 ksi.

In an exemplary experimental embodiment, as illustrated in FIG. 37b, in step 3702, the expandable tubular member 3702a includes a microstructure that includes pearlite and pearlite striation.

In an exemplary embodiment, the expandable tubular member 3702a is then heated at a temperature of 790° C. for about 10 minutes in step 3704.

In an exemplary embodiment, the expandable tubular member 3702a is then quenched in water in step 3706.

In an exemplary experimental embodiment, as illustrated in FIG. 37c, following the completion of step 3706, the expandable tubular member 3702a includes a microstructure that includes ferrite, martensite, and bainite. In an exemplary experimental embodiment, following the completion of step 3706, the expandable tubular member 3702a has a yield strength of 82 ksi, and a tensile strength of 130 ksi.

In an exemplary embodiment, the expandable tubular member 3702a is then radially expanded and plastically deformed using one or more of the methods and apparatus described above. In an exemplary embodiment, following the radial expansion and plastic deformation of the expandable tubular member 3702a, the yield strength of the expandable tubular member is about 130 ksi.

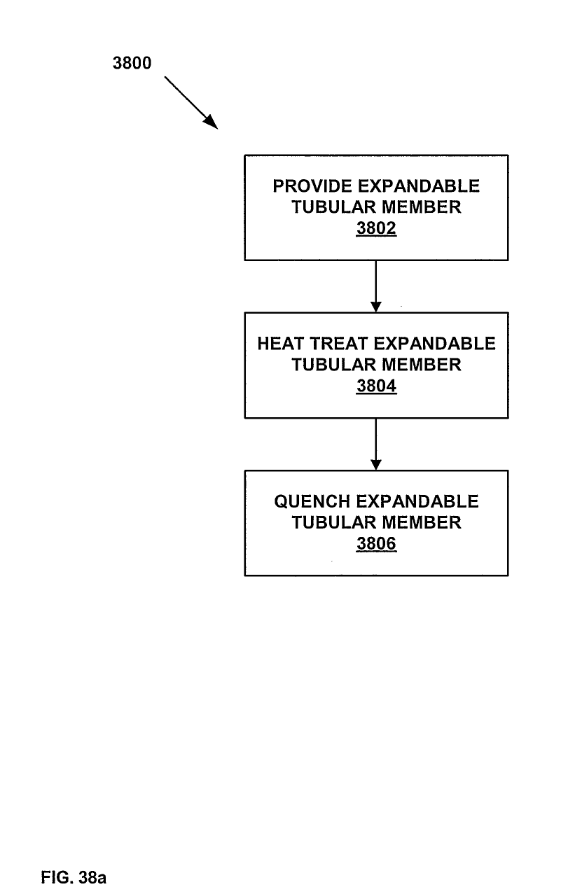

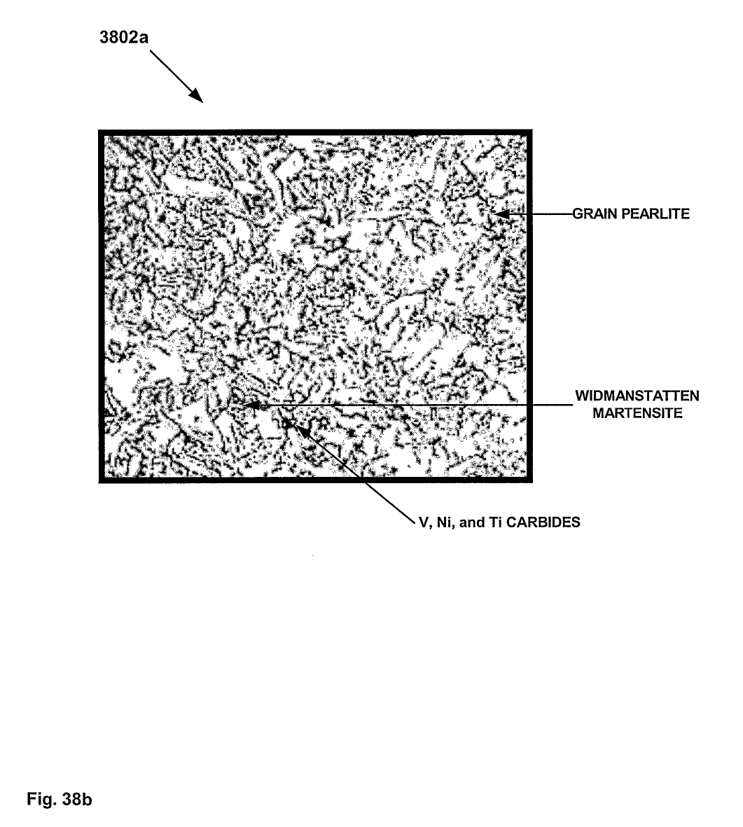

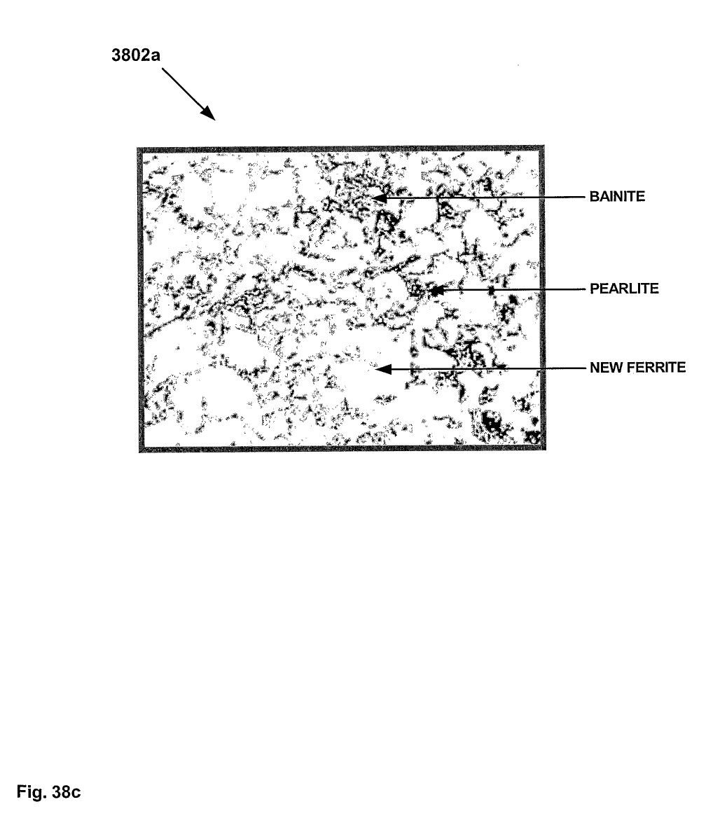

In an exemplary experimental embodiment, as illustrated in FIGS. 38a-38c, one or more of the expandable tubular members, 12, 14, 24, 26, 102, 104, 106, 108, 202, 204 and/or 3502 are processed in accordance with a method 3800, in which, in step 3802, an expandable tubular member 3802a is provided that is a steel alloy having following material composition (by weight percentage): 0.08% C, 0.82% Mn, 0.006% P, 0.003% S, 0.30% Si, 0.06% Cu, 0.05% Ni, 0.05% Cr, 0.03% V, 0.03% Mo, 0.01% Nb, and 0.01% Ti. In an exemplary experimental embodiment, the expandable tubular member 3802a provided in step 3802 has a yield strength of 56 ksi, and a tensile strength of 75 ksi.

In an exemplary experimental embodiment, as illustrated in FIG. 38b, in step 3802, the expandable tubular member 3802a includes a microstructure that includes grain pearlite, widmanstatten martensite and carbides of V, Ni, and/or Ti.

In an exemplary embodiment, the expandable tubular member 3802a is then heated at a temperature of 790° C. for about 10 minutes in step 3804.

In an exemplary embodiment, the expandable tubular member 3802a is then quenched in water in step 3806.

In an exemplary experimental embodiment, as illustrated in FIG. 38c, following the completion of step 3806, the expandable tubular member 3802a includes a microstructure that includes bainite, pearlite, and new ferrite. In an exemplary experimental embodiment, following the completion of step 3806, the expandable tubular member 3802a has a yield strength of 60 ksi, and a tensile strength of 97 ksi.

In an exemplary embodiment, the expandable tubular member 3802a is then radially expanded and plastically deformed using one or more of the methods and apparatus described above. In an exemplary embodiment, following the radial expansion and plastic deformation of the expandable tubular member 3802a, the yield strength of the expandable tubular member is about 97 ksi.

In several exemplary embodiments, the teachings of the present disclosure are combined with one or more of the teachings disclosed in FR 2 841 626, filed on Jun. 28, 2002, and published on Jan. 2, 2004, the disclosure of which is incorporated herein by reference.

A method of determining the expandability of a selected tubular member has been described that includes determining an anisotropy value for the selected tubular member, determining a strain hardening value for the selected tubular member; and multiplying the anisotropy value times the strain hardening value to generate an expandability value for the selected tubular member. In an exemplary embodiment, an anisotropy value greater than 0.12 indicates that the tubular member is suitable for radial expansion and plastic deformation. In an exemplary embodiment, the tubular member includes a wellbore casing, a pipeline, or a structural support.

A method of radially expanding and plastically deforming tubular members has been described that includes selecting a tubular member; determining an anisotropy value for the selected tubular member; determining a strain hardening value for the selected tubular member; multiplying the anisotropy value times the strain hardening value to generate an expandability value for the selected tubular member; and if the anisotropy value is greater than 0.12, then radially expanding and plastically deforming the selected tubular member. In an exemplary embodiment, the tubular member includes a wellbore casing, a pipeline, or a structural support. In an exemplary embodiment, radially expanding and plastically deforming the selected tubular member includes: inserting the selected tubular member into a preexisting structure; and then radially expanding and plastically deforming the selected tubular member. In an exemplary embodiment, the preexisting structure includes a wellbore that traverses a subterranean formation.

A method of selecting tubular members for radial expansion and plastic deformation has been described that includes: selecting a tubular member from a collection of tubular member; determining a carbon content of the selected tubular member; determining a carbon equivalent value for the selected tubular member; and if the carbon content of the selected tubular member is less than or equal to 0.12 percent and the carbon equivalent value for the selected tubular member is less than 0.21, then determining that the selected tubular member is suitable for radial expansion and plastic deformation.

A method of selecting tubular members for radial expansion and plastic deformation has been described that includes: selecting a tubular member from a collection of tubular member; determining a carbon content of the selected tubular member; determining a carbon equivalent value for the selected tubular member; and if the carbon content of the selected tubular member is greater than 0.12 percent and the carbon equivalent value for the selected tubular member is less than 0.36, then determining that the selected tubular member is suitable for radial expansion and plastic deformation.

It is understood that variations may be made in the foregoing without departing from the scope of the invention. For example, the teachings of the present illustrative embodiments may be used to provide a wellbore casing, a pipeline, or a structural support. Furthermore, the elements and teachings of the various illustrative embodiments may be combined in whole or in part in some or all of the illustrative embodiments. In addition, one or more of the elements and teachings of the various illustrative embodiments may be omitted, at least in part, and/or combined, at least in part, with one or more of the other elements and teachings of the various illustrative embodiments.

Although illustrative embodiments of the invention have been shown and described, a wide range of modification, changes and substitution is contemplated in the foregoing disclosure. In some instances, some features of the present invention may be employed without a corresponding use of the other features. Accordingly, it is appropriate that the appended claims be construed broadly and in a manner consistent with the scope of the invention.

1. A method of selecting a tubular member based on its suitability for expansion, comprising:
determining an anisotropy value for a tubular member,
determining a strain hardening value for the tubular member;
multiplying the anisotropy value times the strain hardening value to generate an expandability value for the tubular member; and
selecting the tubular member for expansion based on the generated expandability value.

2. The method of claim 1, wherein an expandability value greater than 0.12 indicates that the tubular member is suitable for radial expansion and plastic deformation.

3. The method of claim 1, wherein the tubular member comprises a wellbore casing.

4. The method of claim 1, wherein the tubular member comprises a pipeline.

5. The method of claim 1, wherein the tubular member comprises a structural support.

6. A method of radially expanding and plastically deforming tubular members, comprising:
selecting a tubular member;
determining an anisotropy value for the selected tubular member;
determining a strain hardening value for the selected tubular member;
multiplying the anisotropy value times the strain hardening value to generate an expandability value for the selected tubular member; and
if the expandability value is greater than 0.12, then radially expanding and plastically deforming the selected tubular member.

7. The method of claim 6, wherein the tubular member comprises a wellbore casing.

8. The method of claim 6, wherein the tubular member comprises a pipeline.

9. The method of claim 6, wherein the tubular member comprises a structural support.

10. The method of claim 6, wherein radially expanding and plastically deforming the selected tubular member comprises,
inserting the selected tubular member into a preexisting structure; and
then radially expanding and plastically deforming the selected tubular member.

11. The method of claim 10, wherein the preexisting structure comprises a wellbore that traverses a subterranean formation.

12. A method of selecting tubular members for radial expansion and plastic deformation, comprising:
selecting a tubular member from a collection of tubular members;
determining a carbon content of the selected tubular member;
determining a carbon equivalent value for the selected tubular member, and
if the carbon content of the selected tubular member is less than or equal to 0.12 percent and the carbon equivalent value for the selected tubular member is less than 0.21, then determining that the selected tubular member is suitable for radial expansion and plastic deformation.

13. A method of selecting tubular members for radial expansion and plastic deformation, comprising:
selecting a tubular member from a collection of tubular members;
determining a carbon content of the selected tubular member;
determining a carbon equivalent value for the selected tubular member; and
if the carbon content of the selected tubular member is greater than 0.12 percent and the carbon equivalent value for the selected tubular member is less than 0.36, then determining that the selected tubular member is suitable for radial expansion and plastic deformation.
